(19) United States
(12) Patent Application Publication
(10) Pub. No.: US 2026/0317839 A1
(43) Pub. Date: Oct. 15, 2026

(54) QUANTITATIVE TRAIT LOCUS ASSOCIATED WITH CBGA DOMINANCE IN CANNABIS

(71) Applicant: Puregene AG, Zeiningen (CH)

(72) Inventors: Claudio Cropano, Zeiningen (CH); Dániel Árpád Carrera, Zeiningen (CH); Gavin Mager George, Zeiningen (CH); Julian Koschmieder, Zeiningen (CH); Leron Katsir, Zeiningen (CH); Maximilian Moritz Vogt, Zeiningen (CH); Michael Edward Ruckle, Zeiningen (CH); Michele Wyler, Zeiningen (CH)

(21) Appl. No.: 19/117,378

(22) PCT Filed: Oct. 3, 2023
(86) PCT No.: PCT/IB2023/059910

§ 371 Date: Mar. 31, 2025

(30) Foreign Application Priority Data
Oct. 3, 2022 (GB) 2214536.1

(51) Int. Cl. C12Q 1/6895; A01H 6/28

(52) U.S. Cl. CPC C12Q 1/6895; A01H 6/28; C12Q 2600/13; C12Q 2600/156

(57) ABSTRACT

The invention relates to methods of identifying a Cannabis spp. plant comprising a quantitative trait locus (QTL) associated with a high CBGA trait, and to Cannabis spp. plants having a high CBGA trait comprising defined allelic states of polymorphisms defining the QTL. Also provided are plants with a high CBGA trait, including a low terpene/cannabinoid trait, identified by the methods described herein. The invention further relates to marker assisted selection and marker assisted breeding methods for obtaining plants having a high CBGA trait, as well as to methods of producing Cannabis spp. plants with the high CBGA trait and plants produced by these methods, based on the allelic state of the QTLs. Further provided are plant extracts having high CBGA levels, and low terpene/cannabinoid levels, obtained from said plants.

### BACKGROUND OF THE INVENTION

The invention relates to methods of identifying a Cannabis spp. plant comprising a quantitative trait locus (QTL) associated with a high CBGA trait and to Cannabis spp. plants having a high CBGA trait. The high CBGA trait is further associated with a low terpene/cannabinoid trait. The invention also relates to marker assisted selection and marker assisted breeding methods for obtaining plants having a high CBGA trait, and a high CBGA trait. Also provided are methods of producing Cannabis spp. plants with the high CBGA trait and high CBGA trait, and plants produced by these methods, as well as plant extracts having high CBGA levels, and low terpene/cannabinoid levels, obtained from said plants.

Modern cannabis is the cross hybridization of three biotypes; Cannabis sativa L. ssp. indica, Cannabis sativa L. ssp. sativa, and Cannabis sativa L. ssp. ruderalis. Cannabis was divergently bred into two distinct, albeit tentative types, based on application. Hemp is primarily used for industrial purposes in feed, food, seed, fiber and oil production. Conversely, high-resin-type (HRT) cannabis is largely cultivated and bred for high concentrations of the pharmacological constituents, cannabinoids, derived from resin in the trichomes. However, recent interest from industrial producers in valuable, novel varieties calls for the convergence of these two types.

Cannabis is the only species in the plant kingdom to produce phytocannabinoids. Phytocannabinoids are a class of terpenoid acting as antagonists and agonists of mammalian endocannabinoid receptors. The pharmacological action is derived from this ability of phytocannabinoids to disrupt and mimic endocannabinoids. Due to its psychoactive properties, one cannabinoid, delta-9-tetrahydrocannabinol (THC), the decarboxylation product of the plant-produced delta-9-tetrahydrocannabinolic acid (THCA), has received much attention in illegal or unregulated breeding programs, with modern HRT varieties having THC concentrations of 0.5% to 30%.

The mechanism by which CBGA is synthesized was proposed by Lou et al (2019), based on in situ reconstitution of the cannabinoid pathway in yeast but has not been demonstrated with in vitro enzyme assays or in vivo in Cannabis sativa tissues. The starting polyketide is hexanoic acid, a breakdown product of fatty acid metabolism, containing a C5 alkyl sidechain. Hexanoic acid is converted into an activated thioester, hexanoyl-CoA, in a reaction catalyzed by acyl activating enzyme 1 (AAE1). In the Olivetolic Acid Cannabinoid Biosynthetic Pathway (OACB Pathway), Hexanoyl-CoA is subsequently lengthened with a malonyl-CoA by olivetol synthase (OLS) (a polyketide synthase (PKS)) followed by a cyclization step by olivetolic acid cyclase (OAC) to produce olivetolic acid (OA). Geranyl pyrophosphate (GPP) from the MEP pathway, together with CBGAS (a prenyltransferase 4 (PT4)) then prenylates OA to form C21 CBGA.

CBGA is the precursor of CBCA CBDA and THCA. They are synthesized by the cannabinoid synthases, CBCAS, CBDAS and THCAS, respectively. It is unclear if the cannabinoid synthases produce only their respective cannabinoids. It has been speculated, for example, that CBDA synthase produces one THCA molecule for every 20 CBDA molecules as an aberrant by-product. This is, up to this point, only supported by their protein sequences and structural similarities. Cannabinoid biosynthesis occurs primarily in the glandular trichomes on cannabis flowers. CBGA is thought to move through the endoplasmic reticulum to be eventually exported to the storage cavity of glandular trichome, where the cannabinoid synthases act to synthesize THCA, CBDA, and CBCA. If the transport of CBGA to the storage cavity were disrupted the cannabinoid synthases would not be able to act on CBGA, leading to its accumulation and possible inhibition of terpene biosynthesis through a yet unknow mechanism.

Glandular trichomes in Cannabis are also a major site of terpene production, compounds that are associated with contributing to the flavour and medicinal qualities of cannabis. Terpenes have many commercial and industrial applications, including as flavour components in beer, and as fragrances in cosmetics. Pharmacological effects of terpenes are an active area of research, with reported effects in humans, including anxiolytic, antibacterial, anti-inflammatory, and sedative effects. In cannabis the interplay between terpenes and cannabinoids has been suggested to create an entourage effect, whereby the compounds enhance and modify each other's effect.

The biosynthetic pathways for terpene and cannabinoid biosynthesis share a common precursor, GPP a product of the MEP pathway. The MEP pathway is plastid localized. GPP is the substrate for the large and diverse terpene synthase family responsible for the vast diversity of mono-terpenes. Sesquiterpenes are also found in cannabis. Sesquiterpene biosynthesis depends on the biosynthesis of the precursor, farnesyl pyrophosphate (FPP), through an enzymatic pathway localized primarily to the peroxisome.

The contribution of terpenes to Cannabis character is poorly understood. Evidence suggest that terpenes may contribute to appealing or non-appealing Cannabis flavour profiles as well as enhancing or modulating the pharmacological effects of cannabinoid, the so called entourage effect. Generally, the global market for Cannabis values terpenoids as adding value to cannabis flowers. We reason that this is generally due to a misconception about terpenes contribution to Cannabis flavour. Cannabis flower with lower levels of terpenes may in fact have enhanced flavour profiles, pharmacological effects that are free from entourage effects may be useful, in addition the lack of terpenes may lead to a shift in abundance of rare cannabinoids, as well as changing the physical properties of the trichomes where they are produced and stored. Oxidation of some terpenes in post-harvest storage can also lead to damage of plant tissue, posing flower quality problems. Interestingly terpenes can also act as attractants of unwanted pests that can damage crops and impact negatively on yield. In addition, low terpene and/or cannabinoid levels may be important breeding target in hemp varieties where the adhesive properties of cannabis leaf and flower can gum up harvesters and other downstream processing machinery due to the high amounts of resin in cannabis. This resin is dominated by cannabinoid and terpene in cannabis.

The industrial hemp industry suffers from over restrictive regulation that set the maximum levels for THC. For example in the United States the 2018 Farm Bill sets the legal limit of tetrahydrocannabinol (THC) to 0.3% or less on a dry weight. Many industrial hemp varieties suffer from exceeding this limit either due to unstable genetic backgrounds and environmental fluctuations. This can lead to farmers not growing hemp as a crop due to the risk and in the worst case farmers losing their crop due to it not being in compliance with legal THC limits. Identifying markers for the introduction of CBGA dominance into exceptional hemp varieties that suffer from running hot can mitigate the risk of cultivating industrial hemp and allow its widespread adoption.

Identifying and selecting for cannabis plants with high CBGA levels, together with low terpene and/or cannabinoid levels, is a novel and important segment for the recreational and pharmacological Cannabis market as well as an important agronomic trait for yield and post-harvest storage.

In the present invention, cannabis plants with increased CBGA levels, together with decreased terpene and cannabinoid levels are provided, together with polymorphisms associated with such increased CBGA levels (and decreased terpene and cannabinoid levels and genetic markers for identifying these plants.

### SUMMARY OF THE INVENTION

The present invention describes methods of identifying and producing a Cannabis spp. plant comprising quantitative trait locus (QTL) associated with a high CBGA trait, wherein the high CBGA trait is associated with low terpene and cannabinoid levels and low adhesion properties. The invention also relates to plants having high CBGA and low terpene/cannabinoid phenotype identified or produced by the methods. The invention further relates to marker assisted selection and marker assisted breeding methods for obtaining plants that have a high CBGA trait, as well as to isolated nucleic acids comprising a quantitative trait locus associated with the trait. Also provided are plant extracts obtained from plants having the high CBGA trait.

According to a first aspect of the present invention there is provided for a method for identifying a Cannabis spp. plant having a high CBGA trait, the method comprising the steps of: (i) genotyping at least one plant with respect to at least one high CBGA QTL by detecting one or more polymorphisms associated with the high CBGA trait as defined in Table 2, and further in Table 3; and (ii) identifying the plant as having the at least one high CBGA QTL based on the genotype at the polymorphism, wherein the at least one high CBGA QTL is associated with the high CBGA trait.

In a first embodiment of the method for identifying a Cannabis spp. plant having a high CBGA trait, the at least one high CBGA QTL may also be associated with a low terpene/cannabinoid trait in the plant.

According to a second embodiment of the method for identifying a Cannabis spp. plant having a high CBGA trait, the polymorphism may be “common_4934”, as defined in Table 2 and further in Table 3. In the homozygous state, Allele 1 as defined in Table 2 (AA), polymorphism “common_4934” has been shown to have particularly high predictive value for the high CBGA trait where CBGA levels are above 2 (% w/w) and cannabinoid and terpene levels are greatly decreased, but the polymorphism “common_4934” is also useful for identifying plants that are heterozygous, carrying Allele 2 as defined in Table 2 (AG), where terpene levels, although lower than plants that are homozygous, i.e., carrying Allele 3 as defined in Table 2 (GG), are not practically eliminated but where these heterozygous plants also have increased CBGA levels in the ranges from 0.15-2 (% w/w) together with decreased cannabinoid levels. This is compared to plants that are homozygous (GG) where CBGA levels rarely are higher than 0.08 (% w/w).

In a third embodiment of the method for identifying a Cannabis spp. plant having a high CBGA trait, the genotyping may be performed by any PCR-based detection method using molecular markers, by sequencing of PCR products containing the one or more polymorphisms, by targeted resequencing, by whole genome sequencing, or by restriction-based methods, for detecting the one or more polymorphisms.

In a fourth embodiment of the method for identifying a Cannabis spp. plant having a high CBGA trait, the molecular markers may be for detecting polymorphisms at regular intervals within the at least one high CBGA QTL such that recombination can be excluded. In an alternative embodiment, the molecular markers may be for detecting polymorphisms at regular intervals within the at least one high CBGA QTL such that recombination can be quantified to estimate linkage disequilibrium between a particular polymorphism and the high CBGA trait. It will be appreciated by those of skill in the art that several possible markers may be designed for detecting the polymorphisms. For example, molecular markers may be for detecting polymorphisms such that recombination events can be detected to a resolution of 10'000 or 100'000 or 500'000 base pairs within the QTL. In one embodiment, the molecular markers may be designed based on a context sequence as defined in Table 3 or may be selected from the primer pairs as defined in Table 4.

In a fifth embodiment of the method for identifying a Cannabis spp. plant having a high CBGA trait, the high CBGA QTL may be a quantitative trait locus having a sequence that corresponds to nucleotides 55368831-61932481 of NC_044378.1 with reference to the CS10 genome defined by one or more polymorphism selected from the group consisting of “common_4934”, “common_4931” and “common_4897” associated with high CBGA and low terpene/cannabinoid levels as defined in Table 2, or a genetic marker linked to the QTL.

According to a second aspect of the present invention, there is provided for a method of producing a Cannabis spp. plant having a high CBGA trait, the method comprising the steps of: (i) providing a donor parent plant having in its genome at least one high CBGA QTL characterized by one or more polymorphisms associated with the high CBGA trait as defined Table 2, or as defined further in Table 3; (ii) crossing the donor parent plant having the at least one high CBGA QTL with at least one recipient parent plant to obtain a progeny population of cannabis plants; (iii) screening the progeny population of cannabis plants for the presence of the at least one high CBGA QTL; and (iv) selecting one or more progeny plants having the at least one high CBGA QTL, wherein the mature plant displays the high CBGA trait.

In a first embodiment of the method of producing a Cannabis spp. plant having a high CBGA trait, the method may further comprise the steps of: (v) crossing the one or more progeny plants with the donor recipient plant; or (vi) selfing the one or more progeny plants.

According to a second embodiment of the method of producing a Cannabis spp. plant having a high CBGA trait, the screening may comprise genotyping at least one plant from the progeny population with respect to the at least one high CBGA QTL by detecting one or more polymorphisms associated with the high CBGA trait as defined Table 2.

In a third embodiment of the method of producing a Cannabis spp. plant having a high CBGA trait, the method my comprise a step of genotyping the donor parent plant with respect to the at least one high CBGA QTL by detecting one or more polymorphisms associated with the high CBGA trait as defined Table 2, or further in Table 3.

According to a fourth embodiment of the method of producing a Cannabis spp. plant having a high CBGA trait, the genotyping may be performed by a PCR-based detection using molecular markers, by sequencing of PCR products containing the one or more polymorphisms, by targeted resequencing, by whole genome sequencing, or by restriction-based methods, for detecting the one or more polymorphisms.

In a fifth embodiment of the method of producing a Cannabis spp. plant having a high CBGA trait, the molecular markers may be for detecting polymorphisms at regular intervals within the high CBGA QTL such that recombination can be excluded. In an alternative embodiment, the molecular markers may be for detecting polymorphisms at regular intervals within the high CBGA QTL such that recombination can be quantified to estimate linkage disequilibrium between a particular polymorphism and the high CBGA trait. For example, molecular markers may be for detecting polymorphisms such that recombination events can be detected to a resolution of 10'000 or 100'000 or 500'000 base pairs within the QTL. It will be appreciated by those of skill in the art that several possible markers may be designed for detecting the polymorphisms. In one embodiment, the molecular markers may be designed based on a context sequence for the polymorphism provided in Table 3 or may be selected from the primer pairs as defined in Table 4.

According to a further embodiment of the method of producing a Cannabis spp. plant having a high CBGA trait, the at least one high CBGA QTL may also be associated with a low terpene/cannabinoid trait in the plant. Of particular use in producing a Cannabis spp. plant having a high CBGA trait, is the polymorphism “common_4934”, as defined in Table 2, and further in Table 3, which has been shown to have particularly high predictive value for the high CBGA QTL and trait. In the homozygous state, Allele 1 as defined in Table 2 (AA), polymorphism “common_4934” has been shown to have particularly high predictive value for the high CBGA trait where CBGA levels are above 2 (% w/w) and cannabinoid and terpene levels are greatly decreased, but the polymorphism “common_4934” is also useful for identifying plants that are heterozygous, carrying Allele 2 as defined in Table 2 (AG), where terpene levels, although lower than plants that are homozygous, i.e., carrying Allele 3 as defined in Table 2 (GG), are not practically eliminated but where these heterozygous plants also have increased CBGA levels in the ranges from 0.15-2 (% w/w) together with decreased cannabinoid levels. This is compared to plants that are homozygous (GG) where CBGA levels rarely are higher than 0.08 (% w/w).

According to a third aspect of the present invention there is provided for a method of producing a Cannabis spp. plant that has a high CBGA trait, the method comprising introducing at least one high CBGA QTL characterized by one or more polymorphisms associated with the high CBGA trait as defined in Table 2 into a Cannabis spp. plant, wherein said high CBGA QTL is associated with the high CBGA trait in the plant.

In one embodiment, introducing the at least one high CBGA QTL may comprise crossing a donor parent plant having the at least one high CBGA QTL characterized by one or more polymorphisms associated with the high CBGA trait with a recipient parent plant. In an alternative embodiment, introducing the at least one high CBGA QTL characterized by one or more polymorphisms associated with the high CBGA trait comprises genetically modifying the Cannabis spp. plant. Several methods of genetic modification are known to those of skill in the art, including targeted mutagenesis, genome editing, and gene transfer. For example, one or more of the polymorphisms associated with the high CBGA trait as defined in Table 2, and further in Table 3, herein may be introduced into a plant by mutagenesis and/or gene editing. In particular the methods of genetically modifying a plant may be selected from the group consisting of CRISPR-Cas9 targeted gene editing, heterologous gene expression using various expression cassettes; TILLING, and non-targeted chemical mutagenesis using e.g., EMS. For example, CRISPR-Cas9 targeted gene editing may be achieved using a guide RNA. Alternatively, a cannabis spp. plant may be transformed with a cassette containing the high CBGA QTL or a part thereof, via any transformation method known in the art.

In a one embodiment of the method of producing a Cannabis spp. plant that has a high CBGA trait, the high CBGA QTL is a quantitative trait locus having a sequence that corresponds to nucleotides 55368831-61932481 of NC_044378.1 with reference to the CS10 genome defined by one or more polymorphism selected from the group consisting of “common_4934”, “common 4931” and “common_4897” associated with high CBGA levels as defined in Table 2, and further in Table 3, or a genetic marker linked to the QTL.

According to a fourth aspect of the present invention there is provided for a Cannabis spp. plant identified according to the method for identifying a Cannabis spp. plant having a high CBGA trait as described herein, provided that the plant is not exclusively obtained by means of an essentially biological process.

In a fifth aspect of the present invention there is provided for a Cannabis spp. plant produced according to the method of a producing a Cannabis spp. plant having a high CBGA trait as described herein. In some embodiments, the Cannabis spp. plant produced according to the method of a producing a Cannabis spp. plant having a high CBGA trait as described herein is not exclusively obtained by means of an essentially biological process.

According to a further aspect of the present invention there is provided for a Cannabis spp. plant comprising at least one high CBGA QTL characterized by one or more polymorphisms associated with a high CBGA trait as defined in Table 2, and further in Table 3. In some embodiments, the plant is not exclusively obtained by means of an essentially biological process.

According to another aspect of the present invention there is provided for an isolated nucleic acid comprising a quantitative trait locus that controls a high CBGA trait in Cannabis spp., wherein the nucleic acid comprising the quantitative trait locus has a sequence that corresponds to nucleotides 55368831-61932481 of NC_044378.1 with reference to the CS10 genome defined by one or more polymorphism selected from the group consisting of “common_4934”, “common 4931” and “common_4897” associated with high CBGA and low terpene/cannabinoid levels as defined in Table 2, or a genetic marker linked to the QTL.

According to yet a further aspect of the present invention there is provided for a Cannabis spp. plant comprising a quantitative trait locus defined herein.

In another aspect there is provided for a plant extract obtained from a Cannabis spp. plant described herein.

In one embodiment of the plant extract, the plant extract may have a cannabinoid and terpene profile with minimum and maximum cannabinoid and terpene ranges as set out for a CBGA dominant plant in Table 1. In particular, the plant, as measured in the dried flower, may have a CBGA content of more than about 2 (% w/w), for example between about 2 and about 10 (% w/w), or a mean CBGA content of about to about 5 (% w/w). The plant may have a total terpene content of less than about 0.25, for example about 0.00 to about 0.25 (% w/w), or a mean total terpene content of about 0.05 (% w/w). The plant may further have a CBDA content of less than about 1 (% w/w), for example between about 0.05 to about 1 (% w/w), or a mean CBDA content of about 0.23 (% w/w). The plant may further have a CBCA content of less than about 1.25 (% w/w), for example between about 0.06 to about 1.25 (% w/w), or a mean CBCA content of about 0.3 (% w/w). The plant may further have a THCA content of less than about 0.2 (% w/w), for example between about 0.00 to about 0.2 (% w/w), or a mean THCA content of about 0.06 (% w/w).

### BRIEF DESCRIPTION OF THE FIGURES

Non-limiting embodiments of the invention will now be described by way of example only and with reference to the following figures:

FIG. 1: Individual terpene concentrations (% w/w) in plants determined to be CBGA Dominant (left bar) or Recessive (right bar). Only terpenes with a mean above 0.0008 are displayed. This excludes the terpenes: sabinene, 3-carene, alpha-phellandrene, alpha-terpinene, beta-ocimene (2 isomers), gamma-terpinene, p-cymene, terpinolene, fenchone, acetic acid, limonene-1.2-epoxide, sabinene hydrate, camphor, linalyl acetate, alpha-cedrene, isopulegol, citral (4 isomers), terpinene-4-ol, menthol, pulegone, isoborneol, borneol, valencene, neryl acetate, beta-bisabolene, geranyl acetate, citronellol, nerol, geraniol, caryophyllene oxide, cis-nerolidol, trans-nerolidol, cedrol, alpha-eudesmol, beta-eudesmol, phytol (2 isomers).

FIG. 2: Relationship between CBGA and terpene amount in the F2 populations tested. The Y-axis shows total terpene amount as (% w/w) and the X-axis shows CBGA amount (% w/w). Block dots are determined to be CBGA dominant plants, triangles are CBGA recessive plants.

FIG. 3: Relationship between CBGA and monoterpene amount and between CBGA and sesquiterpene amount in the F2 populations tested. The Y-axis shows either total mono- or sesqui-terpene amount as (% w/w) and the X-axis shows CBGA amount (% w/w).

FIG. 4: A Manhattan plot showing the results of a GWA for CBGA being used as a proxy for terpene and cannabinoid amount. Each box represents a separate chromosome, with the chromosome name above the box and the position (in base pairs) on the chromosome on the X-axis below, the Y-axis is the LOD score, −log 10(p).

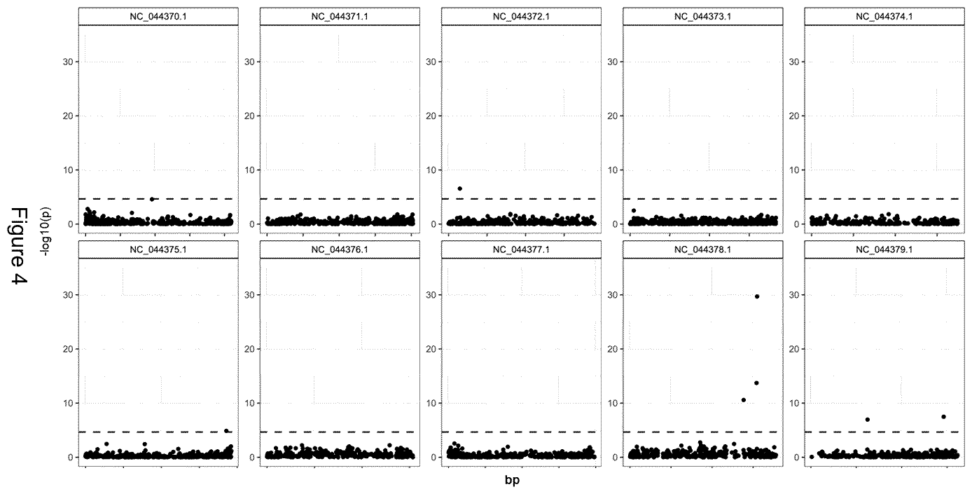

FIG. 5: Allele specific assays to test the SNP marker identified in discriminating the high CBGA trait. Testing KASP3 and common_4934 for predictiveness of the concentration of terpene (% W/W) and CBGA (% W/W). The results of the assays for Allele 1, Allele 2 and the Heterozygous state are shown for KASP3 and common_4934.

### SEQUENCES

The nucleic acid and amino acid sequences listed herein and in any accompanying sequence listing are shown using standard letter abbreviations for nucleotide bases, and the standard one or three letter abbreviations for amino acids. It will be understood by those of skill in the art that only one strand of each nucleic acid sequence is shown, but that the complementary strand is included by any reference to the displayed strand.

### DETAILED DESCRIPTION OF THE INVENTION

The present invention will now be described more fully hereinafter with reference to the accompanying drawings, in which some, but not all embodiments of the invention are shown.

The invention as described should not be limited to the specific embodiments disclosed and modifications and other embodiments are intended to be included within the scope of the invention. Although specific terms are employed herein, they are used in a generic and descriptive sense only and not for purposes of limitation.

As used throughout this specification and in the claims, which follow, the singular forms “a”, “an” and “the” include the plural form, unless the context clearly indicates otherwise.

The terminology and phraseology used herein is for the purpose of description and should not be regarded as limiting. The use of the terms “comprising”, “containing”, “having” and “including” and variations thereof used herein, are meant to encompass the items listed thereafter and equivalents thereof as well as additional items. It is, however, contemplated as a specific embodiment of the present disclosure that the term “comprising” encompasses the possibility of no further members being present, i.e., for the purpose of such an embodiment “comprising” is to be understood as having the meaning of “consisting of”.

In investigating cannabis plants with high CBGA levels, the inventors of the present invention detected a surprising, previously undetected, effect. Plants in which CBGA levels were highly increased showed a dramatic reduction in terpene levels. Somewhat less surprising was that the plants also had a significant decrease in cannabinoid levels. This dramatic reduction in terpene and cannabinoid levels was accompanied by an equally dramatic decrease in the adhesive quality of the flower, as well as a decrease in the cannabis aroma of the flower. They further investigated this phenomenon and identified several polymorphisms associated with a low terpene/cannabinoid phenotype in a CBGA dominant segregating population. The inventors further identified genetic markers to identify plants with the low terpene/cannabinoid phenotype, providing a method to practically eliminate the amount of mono- and sesquiterpene, eliminate cannabinoid derived from CBGA such as THCA, CBDA, and CBCA, as well as reduce the adhesiveness of Cannabis flower and reduce the aroma of cannabis flower. The marker identified by the inventors outperformed previously identified markers by the inventors as well as other groups that linked CBGA dominance to a polymorphism, THCAS G1064A, in a THCA synthase. The inventors find the hypothesis proposed by Garfinkel et al (2021) to be unlikely due to the broad disruption that CBGA dominance confers, in particular to other cannabinoids such as CBDA and CBCA. Regardless of the biochemical mechanism governing CBGA dominance the authors have identified a highly specific marker for the use in selection for the traits described.

Methods are provided herein for identifying and obtaining plants having a high CBGA trait, using a molecular marker detection technique. The inventors of the present invention have further produced and selected for high CBGA levels and low terpene/cannabinoid levels in cannabis spp. plants by crossing plants with the high CBGA trait with plants that do not display the high CBGA trait. Also demonstrated herein, the inventors were able to use genome wide association (GWA) to identify SNPs associated with the high CBGA trait, these SNPs were verified as genetic markers for identifying plants carrying the trait.

Table 2 herein provides several single nucleotide polymorphisms (SNPs) which define the QTLs associated with a high CBGA trait. In some embodiments one or more of the identified SNPs can be used to incorporate the high CBGA trait from a donor plant, containing one or more of the QTLs associated with the trait, into a recipient plant. For example, the incorporation of the high CBGA phenotype and low terpene/cannabinoid genotype may be performed by crossing a donor parent plant to a recipient parent plant to produce plants containing a haploid genome from both parents. Recombination of these genomes provides F1 progeny where each haploid complement of chromosomes, of the diploid genome, is comprised of genetic material from both parents.

In some embodiments, methods of identifying one or more QTLs that are characterized by a haplotype comprising of a series of polymorphisms in linkage disequilibrium are provided. The QTLs each display limited frequency of recombination within the QTLs. Preferably the polymorphisms are selected from any one of Table 2 herein, representing the high CBGA QTLs. Molecular markers may be designed for use in detecting the presence of the polymorphisms and thus the QTLs. Further, the identified QTL polymorphisms and the associated molecular markers may be used in a cannabis breeding program to predict the high CBGA trait of plants in a breeding population and can be used to produce cannabis plants that display a high CBGA phenotype, and low terpene/cannabinoid phenotype, compared to a control population.

Through the introduction of the high CBGA trait by introgression and successive rounds of backcrossing, fixation, and hybridization that is facilitated by tracking the heterozygous or homozygous state of the CBGA trait through the use of the identified markers, HRT cannabis can be integrated into hemp breeding populations while maintaining the high CBGA trait ensuring low THC amount. Similarly, introgression of the high CBGA trait into hemp varieties that are known to have THC amount above regulated thresholds and successive rounds of backcrossing, fixation, and hybridization that is facilitated by tracking the heterozygous or homozygous state of the CBGA trait through the use of the identified markers can produce industrial hemp lines that can be used commercially or as the basis of a breeding population.

As used herein, reference to a plant or variety with a “high CBGA trait” or “high CBGA phenotype” refers to a plant or variety that has above 2% w/w CBGA at the time of harvest and in post-harvest and which displays a low terpene/cannabinoid trait.

As used herein, reference to a plant or a variety with a “low terpene/cannabinoid trait” or “low terpene/cannabinoid phenotype” refers to a plant or a variety that has a total terpene content that is below 0.25% w/w and a total cannabinoid content that is below 2.5% w/w in a flower during the course of plant growth, at the time of harvest and in post-harvest. In particular, with respect to the cannabinoid content, a plant having a low terpene/cannabinoid trait has a CBDA content of less than about 1 (% w/w), such as between about 0.05 to about 1 (% w/w), or a mean CBDA content of about 0.23 (% w/w), a CBCA content of less than about 1.25 (% w/w), such as between about 0.06 to about 1.25 (% w/w), or a mean CBCA content of about 0.3 (% w/w), and a THCA content of less than about 0.2 (% w/w), such as between about 0.00 to about 0.2 (% w/w), or a mean THCA content of about 0.06 (% w/w).

The term “total terpene” as used herein refers to the total amount of terpene measured from dried flower on a Agilent 8890 GC system equipped with a flame ionization detector (FID) and head space sampler and includes the monoterpenes and sesquiterpenes: alpha-pinene, camphene, beta-pinene, sabinene, 3-carene, beta-myrcene, alpha-phellandrene, alpha-terpinene, D-limonene, eucalyptol, beta-hellandrene, beta-ocimene (2 isomers), gamma-terpinene, p-cymene, terpinolene, fenchone, limonene-1.2-epoxide, abinene hydrate, camphor, linalool, linalyl acetate, alpha-cedrene, isopulegol, fenchol, bornyl acetate, isobornyl acetate, citral (4 isomers), beta-caryophyllene, terpinene-4-ol, menthol, pulegone, all-trans-beta-farnesene, isoborneol, alpha-humulene, alpha-terpineol, borneol, valencene, alpha-farnesene, neryl acetate, beta-bisabolene, geranyl acetate, citronellol, beta-maaliene, alpha-bisabolene, selina-3.7 (11)-diene, nerol, geraniol, caryophyllene oxide, cis-nerolidol, trans-nerolidol, guaiol, epi-gamma-eudesmol, cedrol, alpha-bisabolol, alpha-eudesmol, and beta-eudesmol, phytol (2 isomers).

The content of terpenes or cannabinoids is calculated in % of the dry mass of cannabis flower (% w/w). The total terpene content is calculated as the sum of all of the above listed terpenes (% w/w). Similarly, total monoterpene content and total sesquiterpene content are calculated as the sum of the above listed monoterpenes or sesquiterpenes (% w/w), respectively.

The term “total cannabinoid” as used herein refers to the total amount of CBDA, CBCA and THCA measured from dried flower on a Agilent 8890 GC system equipped with a flame ionization detector (FID) and head space sampler.

The “time of harvest” is defined with respect to the maturity of the flower, where approximately greater than 50% of the pistils have turned brown in appearance. Alternatively, the time of harvest can also be determined by initiation of flowering for hemp-type cannabis or by other agronomic criteria common in the art.

It is a particular aim of the present invention to identify and characterize a plant for the high CBGA trait early in the plant lifecycle, particularly prior to the plant displaying the high CBGA trait. This can be achieved by genotyping the plant using molecular markers for detecting at least one QTL associated with the high CBGA trait.

As used herein a “quantitative trait locus” or “QTL” is a polymorphic genetic locus with at least two alleles that differentially affect the expression of a continuously varying phenotypic trait when present in a plant or organism which is characterised by a series of polymorphisms in linkage disequilibrium with each other.

As used herein, the term “high CBGA QTL” or “high CBGA quantitative trait locus” refers to a quantitative trait locus characterized by one or more polymorphisms having an allelic state associated with the high CBGA trait and low terpene/cannabinoid trait described or defined in Table 2.

As described herein, in one embodiment it is desirable to obtain a plant displaying a high CBGA trait.

As used herein, “haplotypes” refer to patterns or clusters of alleles or single nucleotide polymorphisms that are in linkage disequilibrium and therefore inherited together from a single parent. The term “linkage disequilibrium” refers to a non-random segregation of genetic loci or markers. Markers or genetic loci that show linkage disequilibrium are considered linked.

As used herein, the term “high CBGA haplotype” refers to the subset of the polymorphisms contained within any one of the high CBGA QTLs which exist on a single haploid genome complement of the diploid genome, and which are in linkage disequilibrium with the high CBGA trait.

As used herein, the term “donor parent plant” refers to a plant that is either homozygous or heterozygous for the high CBGA haplotype or which contains one or more of the high CBGA QTLs.

As used herein, the term “recipient parent plant” refers to a plant that is not heterozygous or homozygous for the high CBGA QTLs, or the high CBGA haplotype.

The term “crossed” or “cross” means the fusion of gametes via pollination to produce progeny (e.g., cells, seeds or plants). The term encompasses both sexual crosses (the pollination of one plant by another) and selfing (self-pollination, e.g., when the pollen and ovule are from the same, or genetically identical plant). The term “crossing” refers to the act of fusing gametes via pollination to produce progeny.

The term “high CBGA allele” refers to the haplotype allele within a particular QTL that confers, or contributes to, the high CBGA trait, or alternatively, is an allele that allows the identification of plants with the high CBGA trait, that can be included in a breeding program (“marker assisted breeding” or “marker assisted selection” “or “genomic selection”).

The term “GWAS” or “Genome wide association study” or “GWA” or “Genome wide association” as used herein refers to an observational study of a genome-wide set of genetic variants or polymorphisms in different individual plants to determine if any variant or polymorphism is associated with a trait, specifically the high CBGA trait.

As used herein a “polymorphism” is a particular type of variance that includes both natural and/or induced multiple or single nucleotide changes, short insertions, or deletions in a target nucleic acid sequence at a particular locus as compared to a related nucleic acid sequence. These variations include, but are not limited to, single nucleotide polymorphisms (SNPs), indel/s, genomic rearrangements, gene duplications, as well as genome insertions and deletions.

As used herein, the term “LOD score” or “logarithm (base 10) of odds” refers to a statistical estimate used in linkage analysis, wherein the score compares the likelihood of obtaining the test data if the two loci are indeed linked, to the likelihood of observing the same data purely by chance. The LOD score is a statistical estimate of whether two genetic loci are physically near enough to each other (or “linked”) on a particular chromosome that they are likely to be inherited together. A LOD score of 3 or higher is generally understood to mean that two genes are located close to each other on the chromosome. In terms of significance, a LOD score of 3 means the odds are 1,000:1 that the two genes are linked and therefore inherited together.

As used herein, the term “quantile-quantile” or “Q-Q” refers to a graphical method for comparing two probability distributions by plotting their quantiles against each other. If the two distributions being compared are similar, the points in the Q-Q plot will approximately lie on the line y=x. If the distributions are linearly related, the points in the Q-Q plot will approximately lie on a line, but not necessarily on the line y=x. Q-Q plots can also be used as a graphical means of estimating parameters in a location-scale family of distributions.

As used herein, a “causal gene” is the specific gene having a genetic variant (the “causal variant”) which is responsible for the association signal at a locus and has a direct biological effect on the high CBGA phenotype. In the context of association studies, the genetic variants which are responsible for the association signal at a locus are referred to as the “causal variants”. Causal variants may comprise one or more “causal polymorphisms” that have a biological effect on the phenotype.

The term “nucleic acid” encompasses e both ribonucleotides (RNA) and deoxyribonucleotides (DNA), including cDNA, genomic DNA, isolated DNA and synthetic DNA. The nucleic acid may be double-stranded or single-stranded. Where the nucleic acid is single-stranded, the nucleic acid may be the sense strand or the antisense strand. A “nucleic acid molecule” or “polynucleotide” refers to any chain of two or more covalently bonded nucleotides, including naturally occurring or non-naturally occurring nucleotides, or nucleotide analogs or derivatives. By “RNA” is meant a sequence of two or more covalently bonded, naturally occurring or modified ribonucleotides. The term “DNA” refers to a sequence of two or more covalently bonded, naturally occurring or modified deoxyribonucleotides. By “cDNA” is meant a complementary or copy DNA produced from an RNA template by the action of RNA-dependent DNA polymerase (reverse transcriptase).

In some embodiments, the nucleic acid molecules of the invention may be operably linked to other sequences. By “operably linked” is meant that the nucleic acid molecules, such as those comprising the QTLs of the invention or genes identified herein, and regulatory sequences are connected in such a way as to permit expression of the proteins when the appropriate molecules are bound to the regulatory sequences. Such operably linked sequences may be contained in vectors or expression constructs which can be transformed or transfected into plant cells or plants for expression. A “regulatory sequence” refers to a nucleotide sequence located either upstream, downstream or within a coding sequence. Generally regulatory sequences influence the transcription, RNA processing or stability, or translation of an associated coding sequence. Regulatory sequences include but are not limited to: effector binding sites, enhancers, introns, polyadenylation recognition sequences, promoters, RNA processing sites, stem-loop structures, translation leader sequences and the like.

The term “promoter” refers to a DNA sequence that is capable of controlling the expression of a nucleic acid coding sequence or functional RNA. A promoter may be based entirely on a native gene, or it may be comprised of different elements from different promoters found in nature. Different promoters are capable of directing the expression of a gene at different stages of development, or in response to different environmental or physiological conditions. An “inducible promoter” is promoter that is active in response to a specific stimulus. Several such inducible promoters are known in the art, for example, chemical inducible promoters, developmental stage inducible promoters, tissue type specific inducible promoters, hormone inducible promoters, environment responsive inducible promoters.

The term “isolated”, as used herein means having been removed from its natural environment. Specifically, the nucleic acid or gene(s) identified herein may be isolated nucleic acids or gene(s), which have been removed from plant material where they naturally occur.

The term “purified”, relates to the isolation of a molecule or compound in a form that is substantially free of contamination or contaminants. Contaminants are normally associated with the molecule or compound in a natural environment, purified thus means having an increase in purity as a result of being separated from the other components of an original composition. The term “purified nucleic acid” describes a nucleic acid sequence that has been separated from other compounds including, but not limited to polypeptides, lipids, and carbohydrates which it is ordinarily associated with in its natural state.

The term “complementary” refers to two nucleic acid molecules, e.g., DNA or RNA, which are capable of forming Watson-Crick base pairs to produce a region of double-strandedness between the two nucleic acid molecules. It will be appreciated by those of skill in the art that each nucleotide in a nucleic acid molecule need not form a matched Watson-Crick base pair with a nucleotide in an opposing complementary strand to form a duplex. One nucleic acid molecule is thus “complementary” to a second nucleic acid molecule if it hybridizes, under conditions of high stringency, with the second nucleic acid molecule. A nucleic acid molecule according to the invention includes both complementary molecules.

As used herein a “substantially identical” or “substantially homologous” sequence is a nucleotide sequence that differs from a reference sequence only by one or more conservative substitutions, or by one or more non-conservative substitutions, deletions, or insertions located at positions of the sequence that do not destroy or substantially alter the activity of the polypeptide encoded by the nucleic acid molecule. Alignment for purposes of determining percent sequence identity can be achieved in various ways that are within the knowledge of those with skill in the art. These include using, for instance, computer software such as ALIGN, Megalign (DNASTAR), CLUSTALW or BLAST software. Those skilled in the art can readily determine appropriate parameters for measuring alignment, including any algorithms needed to achieve maximal alignment over the full length of the sequences being compared. In one embodiment of the invention there is provided for a polynucleotide sequence that has at least about 80% sequence identity, at least about 90% sequence identity, or even greater sequence identity, such as about 95%, about 96%, about 97%, about 98% or about 99% sequence identity to the sequences described herein.

Alternatively, or additionally, two nucleic acid sequences may be “substantially identical” or “substantially homologous” if they hybridize under high stringency conditions. The “stringency” of a hybridisation reaction is readily determinable by one of ordinary skill in the art, and generally is an empirical calculation which depends upon probe length, washing temperature, and salt concentration. In general, longer probes required higher temperatures for proper annealing, while shorter probes require lower temperatures. Hybridisation generally depends on the ability of denatured DNA to re-anneal when complementary strands are present in an environment below their melting temperature. A typical example of such “stringent” hybridisation conditions would be hybridisation carried out for 18 hours at 65° C. with gentle shaking, a first wash for 12 min at 65° C. in Wash Buffer A (0.5% SDS; 2×SSC), and a second wash for 10 min at 65° C. in Wash Buffer B (0.1% SDS; 0.5% SSC).

Nucleotide positions of polymorphisms described herein are provided with reference to the corresponding position on the Cannabis sativa (assembly cs10) representative genome, provided as RefSeq assembly accession: GCF_900626175.2 on NCBI, loaded on 14 Feb. 2019, referred to herein as “cs10 reference genome” or “cs10 genome”.

### Methods of Identifying a QTL or Haplotype Responsible for the High CBGA Trait and Molecular Markers Therefor

In some embodiments, methods are provided for identifying a QTL or haplotype responsible for the high CBGA trait and for selecting plants with the high CBGA trait. In some embodiments, the methods may comprise the steps of:
a. Identifying a plant that displays the high CBGA phenotype within a breeding program.
b. Establishing a population by crossing the identified plant to itself (selfing) or a recipient parent plant.
C. Genotyping the resultant F1, or subsequent populations, for example by sequencing methods.
d. Performing association studies, including phenotyping and linkage analysis, to discover QTLs and/or polymorphisms contained within the QTL.
e. Optionally, identifying cannabis paralogs of previously characterized genes that may be involved in the high CBGA phenotype.
f. Developing molecular markers that detect one or more polymorphisms linked to QTLs, alleles within these QTLs, or existing or induced polymorphisms.
g. Validating the molecular markers by determining the linkage disequilibrium between the marker and the high CBGA trait.

### Trait Development and Introgression

In some embodiments, methods are provided for marker assisted breeding (MAB) or marker assisted selection (MAS) of plants having a high CBGA QTL or trait. The methods may comprise the steps of:
a. Identifying a plant that displays the high CBGA trait or phenotype or contains a high CBGA QTL as defined herein.
b. Establishing a population by crossing the identified plant to itself (selfing) or another recipient parent plant.
C. Genotyping and phenotyping the resultant F1, or subsequent, populations, for example by sequencing methods.
d. Performing association studies, inputting phenotype and genotype information to identify genomic regions enriched with polymorphisms associated with the high CBGA trait, to discover QTLs and/or polymorphisms contained within the QTL.
e. Optionally, identifying cannabis paralogs of previously characterized genes that may be involved in the high CBGA phenotype.
f. Developing molecular markers that detect one or more polymorphisms linked to QTLs, alleles within these QTLs, or existing or induced polymorphisms.
g. Using the molecular markers when introgressing the QTLs or polymorphisms into new or existing cannabis varieties to select plants containing the high CBGA haplotype or the high CBGA trait.

### QTLs and Marker Assisted Breeding

In some embodiments, during the breeding process, selection of plants displaying the high CBGA trait may be based on molecular markers designed to detect polymorphisms linked to genomic regions that control the high CBGA trait by either an identified or an unidentified mechanism. Previously identified genetic mechanisms may, for example, have a direct or pleiotropic effect on terpene concentrations in a plant. In some embodiments, QTLs containing such elements are identified using association studies. Knowledge of the mode-of-action is not required for the functional use of these genomic regions in a breeding program. Identification of regions controlling unidentified mechanisms may be useful in obtaining plants with the high CBGA phenotype, based on identification of polymorphisms that are either linked to, or found within QTLs that are associated with the high CBGA phenotype using association studies.

### Construction of Breeding Populations

Breeding populations are the offspring of sexual reproduction events between two or more parents. The parent plants (F0) are crossed to create an F1 population each containing a chromosomal complement of each parent. In a subsequent cross (F2), recombination has occurred and allows for mostly independent segregation of traits in the offspring and importantly the reconstitution of recessive phenotypes that existed in only one of the parental lines.

According to some embodiments, QTLs that lead to the high CBGA phenotype are identified within synthetic populations of plants capable of revealing dominant, recessive, or complex traits. In one embodiment of the invention, a genetically diverse population of cannabis varieties, that are used to produce the synthetic population are integrate them into a breeding program by unnatural processes. In some embodiments, these processes result in changes in the genomes of the plants. The changes may include, but are not limited to, mutations and rearrangements in the genomic sequences, duplication of the entire genome (polyploidy), or activation of movement of transposable elements which may inactivate, activate or attenuate the activity of genes or genomic elements. According to one embodiment of the invention, the following methods employed to integrate the plants into a breeding program include some or all of the following:
a. Growing plants in rich media or soils under artificial lighting;
b. Cloning of plants, often through a multitude of sub-cloning cycles;
C. Introduction of plants into in vitro, sterile growth environments, and subsequent removal to standard growth conditions;
d. Exposure to mutagens such as EMS, colchicine, silver nitrate, ethidium bromide, dinitroanalines, high concentrations mono or poly-chromatic light sources;
e. Growing plants under highly stressful conditions which include restricted space, drought, pathogen challenge, atypical temperatures, and nutrient stresses.

### High CBGA Trait Association Studies and QTL Identification

In some embodiments, the synthetic populations created are either the offspring of the sexual reproduction or clones of plants in the breeding program such that genetic material of individuals in the synthetic populations is derived from one, or two, or more plants from the breeding program.

In one embodiment, plants identified within the synthetic population as having a trait of interest, such as the high CBGA trait, may be used to create a structured population for the identification of the genetic locus responsible for the trait. The structured population may be created by crossing one (selfing) or more plants and recovering the seeds from those plants.

Plants in the structured population may be fully genotyped using genome sequencing to identify genetic markers for use in the association study (AS) database. Association mapping is a powerful technique used to detect quantitative trait loci (QTLs) specifically based on the statistical correlation between the phenotype and the genotype. In this case the trait is the high CBGA phenotype, and low terpene/cannabinoid pheotype. In a population generated by crossing, the amount of linkage disequilibrium (LD) is reduced between genetic marker and the QTL as a function of genetic distance in cannabis varieties with similar genome structures. Simple association mapping is performed by biparental crosses of two closely related lines where one line has a phenotype of interest, and the other does not. In some embodiments, advanced population structures may be used, including nested association mapping (NAM) populations or multi-parent advanced generation inter-cross (MAGIC) populations, however it will be appreciated that other population structures can also be effectively used. Biparental, NAM, or MAGIC structured populations can be generated and offspring, at F1 or later generations, may be maintained by clonal propagation for a desired length of time. In some embodiments, QTLs may be identified using the high-density genetic marker database created by genotyping the founder lines and structured population lines. This marker database may be coupled with an extensive phenotypic trait characterization dataset, including, for example, characterization of the high CBGA phenotype and low terpene/cannabinoid phenotype of the plants. Using the association studies described herein, together with accurate phenotyping, this method is able to identify genomic regions, QTLs and even specific genes or polymorphisms responsible for the high CBGA phenotype that is directly introduced into recipient lines. Polygenic phenotypes may also be identified using the methods described herein.

In one embodiment, the structured population is grown to the flowering stage. To characterize the phenotypes of the lines, they are clonally reproduced so the phenotypic data can be collected in feasible replicates.

### Genomic Selection

In some embodiments, during the breeding process, selection of plants by genomic selection (GS) may be conducted. Genomic selection is a method in plant breeding where the genome wide genetic potential of an individual is determined to predict breeding values for those individuals. In some embodiments, the accuracy of genomic selection is affected by the data used in a GS model including size of the training population, relationships between individuals, marker density, use of pedigree information, and inclusion of known QTLs.

In some embodiments, a QTL or a SNP known to be associated with a trait that contributes to selection criteria can improve the accuracy of genomic selection models. In some embodiments, a genomic selection model that incorporates terpene concentrations can be improved by the inclusion of the high CBGA QTLs in the GS model.

### Molecular Markers to Detect Polymorphisms

As used herein, the term “marker” or “genetic marker” refers to any sequence comprising a particular polymorphism or haplotype described herein that is capable of detection. For example, a marker may be a binding site for a primer or set of primers that is designed for use in a PCR-based method to amplify and thus detect a polymorphism or haplotype.

Alternatively, the marker may introduce a restriction enzyme recognition site, or result in the removal of a restriction enzyme recognition site. Plants can be screened for a particular trait based on the detection of one or more markers confirming the presence of the polymorphism. Marker detection systems that may be used in accordance with the present invention include, but are not limited to polymerase chain reaction (PCR) followed by sequencing, Kompetitive allele specific PCR (KASP), restriction fragment length polymorphisms (RFLPs) analysis, amplified fragment length polymorphisms (AFLPs), cleaved amplified polymorphic sequences (CAPS), or any other markers known in the art.

In some embodiments “molecular markers” refers to any marker detection system and may be PCR primers, or targeted sequencing primers such as those described in the examples below, more specifically the primers defined in Table 4.

For example, PCR primers may be designed that consist of a reverse primer and two forward primers that are homologous to the part of the genome that contains a polymorphism but differ in the 3′ nucleotide such that the one primer will preferentially bind to sequences containing the polymorphism and the other will bind to sequences lacking it. The three primers are used in single PCR reactions where each reaction contains DNA from a cannabis spp. plant as a template. Fluorophores linked to the forward primers provide, after thermocycling, a different relative fluorescent signal for homozygous and heterozygous alleles containing the polymorphism and for those lacking the polymorphism, respectively.

In some embodiments, allele-specific primers may each harbor a unique tail sequence that corresponds with a universal FRET (fluorescence resonant energy transfer) cassette. For example, the primer specific to the SNP may be labelled with a FAM and the other specific primer with a HEX dye. During the PCR thermal cycling performed with these primers, the allele-specific primer binds to the genomic DNA template and elongates, so attaching the tail sequence to the newly synthesized strand. The complement of the allele-specific tail sequence is then generated during subsequent rounds of PCR, enabling the FRET cassette to bind to the DNA. Alleles are discriminated through the competitive binding of the two allele-specific forward primers. At the end of the PCR reaction a fluorescent plate is read using standard tools which may include RT-PCR devices with the capacity to detect florescent signals and is evaluated with commercial software.

If the genotype at a given polymorphism site is homozygous, one of the two possible fluorescent signals will be generated. If the genotype is heterozygous, a mixed fluorescent signal will be generated. By way of example, genomic DNA extracted from cannabis leaf tissue at seedling stage can be used as a template for PCR amplifications with reaction mixtures containing the three primers. Final fluorescent signals can be detected by a thermocycler and analyzed using standard software for this purpose, which discriminates between individuals that are heterozygotes or homozygotes for either allele.

In some embodiments, molecular markers to one, two, or more of the SNPs in the haplotype can be used to identify the presence of the QTL and by association, the high CBGA phenotype and low terpene/cannabinoid phenotype.

Further, the QTL may include a number of individual polymorphisms in linkage disequilibrium, which constitute a haplotype and which, with high frequency, can be inherited from a donor parent plant as a unit. Therefore, in some embodiments, molecular markers can be utilized which have been designed to identify numerous polymorphisms which are in linkage disequilibrium with other polymorphisms, any of which can be used to effectively predict the phenotype of the offspring for the high CBGA trait.

According to some embodiments, any polymorphism in linkage disequilibrium with one or more of the high CBGA QTLs can be used to determine the high CBGA haplotype in a breeding population of plants, as long as the polymorphism is unique to the high CBGA trait in the donor parent plant when compared to the recipient parent plant.

In some embodiments the desired trait is the high CBGA trait, and the donor parent plant may be a plant that has been genetically modified or selected to include a high CBGA QTL defined by a polymorphism conferring the high CBGA trait, and low terpene/cannabinoid trait, for example any, some, or all of the polymorphisms defined in Table 2.

In some embodiments, donor parent plants, as described above, are used as one of two parents to create breeding populations (F1) through sexual reproduction. In this embodiment, donor parent plants may be identified by detecting polymorphisms using the molecular markers as described above.

Methods for reproduction that are known in the art may be used. The donor parent plant provides the high CBGA trait to the breeding population. The trait is made to segregate through the population (F2) through at least one additional crossing event of the offspring of the initial cross. This additional crossing event can be either a selfing of one of the offspring or a cross between two individuals, provided that each plant used in the F1 cross contains at least one copy of a desired QTL allele or haplotype.

In some embodiments, the high CBGA allele or high CBGA haplotype in plants to be used in the F1 cross is determined using the described molecular markers. In some embodiments, the resulting F2 progeny, or subsequent progeny, is/are screened for any of the polymorphisms associated with the high CBGA trait described herein.

The plants at any generation can be produced by asexual means like cutting and cloning, or any method that yields a genetically identical offspring.

Production of Cannabis Spp. Plants Having the High CBGA Trait (Including the Low Terpene/Cannabinoid Trait)

In some embodiments, a Cannabis spp. plant that has a high terpene trait or phenotype may be converted into a plant having a high CBGA trait according to the methods of the present invention by providing a breeding population where the donor parent plant contains an high CBGA QTL associated with a high CBGA trait and recipient parent plant displays a high terpene phenotype.

In some embodiments the high terpene phenotype may be removed from a recipient parent plant by crossing it with a donor parent plant having the high CBGA QTL. In some embodiments the donor parent plant has a high CBGA phenotype and a contains a contiguous genomic sequence characterized by one or more of the polymorphisms of Table 2 associated with the high CBGA allele or haplotype.

In some embodiments, the donor parent plant is any cannabis spp. that is cross fertile with the recipient parent plant.

In some embodiments, MAS or MAB may be used in a method of backcrossing plants carrying the high CBGA trait to a recipient parent plant. For example, an F1 plant from a breeding population can be crossed again to the recipient parent plant. In some embodiments, this method is repeated.

In some embodiments, the resulting plant population is then screened for the high CBGA trait using MAS with molecular markers to identify progeny plants that contain one or more polymorphism, such as any of those described Table 2, indicating the presence of an allele of a QTL associated with the high CBGA phenotype. In another embodiment, the population of cannabis spp. plants may be screened by any analytical methods known in the art to identify plants with desired characteristics.

### Methods to Genetically Engineer Plants to Achieve the High CBGA Phenotype and Low Terpene/Cannabinoid Phenotype Using Mutagenesis or Gene Editing Techniques

Identifying QTLs, and individual polymorphisms, that correlate with a trait when measured in an F1, F2, or similar, breeding population indicates the presence of one or more causative polymorphisms in close proximity the polymorphism detected by the molecular marker. In some embodiments, the polymorphisms associated with the absence or presence, of the high CBGA trait are introduced into a plant by other means so that a trait, such as the high CBGA trait, can be removed from, or introduced into, plants that would otherwise contain associated causative polymorphisms.

The entire QTLs or parts thereof which confer the high CBGA trait, described herein may be introduced into the genome of a cannabis spp. plant to obtain plants with high CBGA (and low terpene/cannabinoid) phenotypes, through a process of genetic modification known in the art, for example, but not limited to, heterologous gene expression using various expression cassettes.

The trait described herein may be removed from, or introduced into, the genome of a cannabis spp. plant to obtain plants that exclude or include the causative polymorphisms and the potential to display a desired high CBGA phenotype, and low terpene/cannabinoid phenotype, through processes of genetic modification known in the art, for example, but not limited to, CRISPR-Cas9 targeted gene editing, TILLING, non-targeted chemical mutagenesis using e.g., EMS.

The present invention further provides methods for producing a modified Cannabis spp. plant using genome editing or modification techniques. For example, genome editing can be achieved using sequence-specific nucleases (SSNs) the use of which results in chromosomal changes, such as nucleotide deletions, insertions or substitutions at specific genetic loci, particularly those associated with high CBGA and low terpene/cannabinoid levels described in Table 2. Non limiting examples of SSNs include zinc finger nucleases (ZFNs), TAL effector nucleases (TALENs), meganuclease, and, clustered regularly interspaced short palindromic repeats (CRISPR)/CRISPR-associated protein (Cas) system. In some embodiments, non-limiting examples of Cas proteins suitable for use in the methods of the present invention include Csnl, Cpf1 Cas9, Cas 12, Cas 13, Cas 14, CasX and combinations thereof. In one embodiment, a modified Cannabis spp. plant having high CBGA and low terpene/cannabinoid levels is generated using CRISPR/Cas9 technology, which is based on the Cas9 DNA nuclease guided to a specific DNA target by a single guide RNA (sgRNA). For example, the genome modification may be introduced using guide RNA, e.g. single guide RNA (sgRNA) designed and targeted to introduce a polymorphism associated with the high CBGA trait as set out in Table 2.

DNA introduction into the plant cells can be performed using Agrobacterium infiltration, virus-based plasmid delivery of the genome editing molecules and mechanical insertion of DNA (PEG mediated DNA transformation, biolistics, etc.). In some embodiments, the Cas9 protein may be directly inserted together with a gRNA (ribonucleoprotein-RNP's) in order to bypass the need for in vivo transcription and translation of the Cas9+gRNA plasmid in planta to achieve gene editing. In one embodiment, a genome edited plant may be developed and used as a rootstock, so that the Cas protein and gRNA can be transported via the vasculature system to the top of the plant and create the genome editing event in the scion.

According to one embodiment of the present invention, the method of genetically modifying a plant may be achieved by combining the Cas nuclease (e.g. Cas9, Cpf 1) with a predefined guide RNA molecule (gRNA). The gRNA is complementary to a specific DNA sequence targeted for editing in the plant genome and which guides the Cas nuclease to a specific nucleotide sequence. The predefined gene specific gRNA's may be cloned into the same plasmid as the Cas gene and this plasmid is inserted into plant cells as described above.

In some embodiments, once the guide RNA molecule and Cas9 nuclease reach the specific predetermined DNA sequence, the Cas9 nuclease cleaves both DNA strands to create double stranded breaks leaving blunt ends. This cleavage site is then repaired by the cellular non homologous end joining DNA repair mechanism resulting in insertions or deletions which introduce a mutation at the cleavage site.

In one embodiment, a deletion form of the mutation may consist of at least 1 base pair deletion. As a result of this base pair deletion the gene coding sequence for the gene responsible for high CBGA and low terpene/cannabinoid concentrations is disrupted and the translation of the encoded protein is compromised either by a premature stop codon or disruption of a functional or structural property of the protein.

In another embodiment, the high CBGA trait in Cannabis spp. plants may be introduced by generating gRNA with homology to a specific site of predetermined genes in the Cannabis spp. genome or the QTLs defined herein. This gRNA may be sub-cloned into a plasmid containing the Cas9 gene, and the plasmid inserted into the Cannabis spp. plant cells. In this way site specific mutations in the QTLs are generated thus effectively reducing total terpene concentrations to below about 0.1 (% w/w) in the genome edited plant.

In some embodiments, a modified Cannabis spp. plant exhibiting a high CBGA phenotype, and low terpene/cannabinoid phenotype, may be obtained using the targeted genome modification methods described above, wherein the plant comprises a targeted genome modification to introduce one or more polymorphisms associated with the high CBGA trait defined in Table 2, wherein the modification effects the high CBGA trait.

Plants may be screened with molecular markers as described herein to identify transgenic individuals with the high CBGA trait or having a high CBGA QTL or polymorphism(s), following the genetic modification.

In some embodiments, cannabis spp. plants having one or more of the polymorphisms of Table 2 associated with the high CBGA trait QTLs or linked thereto are provided. The polymorphisms may be introduced, for example, by genetic engineering. In some embodiments the one or more polymorphisms associated with the high CBGA trait or linked thereto are introduced into the plants by breeding, such as by MAS or MAB, for example as described herein.

The high CBGA QTLs herein, or genes identified herein responsible for effecting the high CBGA trait, may be under the control of, or operably linked to, a promoter, for example an inducible promoter. Such QTLs or genes may be operably linked to the inducible promoter so as to induce or suppress the high CBGA trait or phenotype in the plant or plant cell.

Accordingly, in a further embodiment, Cannabis spp. plants comprising a high CBGA QTL described herein, or one or more polymorphisms associated therewith, are provided. In some cases, such plants are provided for with the proviso that the plant is not exclusively obtained by means of an essentially biological process.

The following examples are offered by way of illustration and not by way of limitation.

### Example 1

### Evaluation of Cannabinoid and Terpene Levels in CBGA Dominant Cannabis

A single nucleotide polymorphism associated with plants that display high amounts of CBGA with lower levels of THCA and CBDA has previously been identified in U.S. Patent Application Publication Number 2022/0228159 A1, which is incorporated in its entirety herein by reference. The SNP, designated as G1064A, is a single nucleotide G/A polymorphism at position 1064 of the THCA synthase gene, which results in an amino acid change from a serine to an asparagine at position 355 of the TCHA synthase enzyme.

The inventors of the present invention reasoned that it was unlikely that the THCAS G1064A SNP, proposed to be associated with the high CBGA phenotype, was causative for the high CBGA/low THCA phenotype and set out to identify the causative polymorphism or other polymorphisms more closely linked to the high CBGA trait. The inventors reasoned that using the F2 populations segregating for the dominant CBGA phenotype they would be able to identify molecular markers associated with high CBGA levels.

To investigate the genetic factors contributing to plants that display high amounts of CBGA with lower levels of THCA and CBDA, several F2 populations were generated by crossing plants heterozygous for CBGA dominance with plants without the CBGA dominant genotype (Table 1). The progeny of these populations were selfed. Populations displaying CBGA dominant plants were used in this study. Flowers that were harvested were used as material to characterize the cannabinoid profile of the plants that comprised the 4 F2 populations, comprising a total of 365 individuals (Table 1).

The F2 populations were grown in a field trial in 2021 and flower was collected at the time of harvest. At the time of harvest, flowers were harvested from the primary flowering stem in mid-October 2021, dried, and analyzed for their constituent cannabinoid content.

Cannabinoid extraction from flower material was performed through mechanical homogenization in a VWR Starbeater mill. Approximately 500 mg of plant flower material (weight noted) and 15 ml ethanol (99.6%, Ph.Eur. grade) were added to a disposable 50 ml test tubes with zirconia beads (~2 mm diameter) and cannabinoids were extracted by shaking for 5 min at 25 Hz. An aliquot of the crude extract was directly filtered through a 0.2 μm PTFE syringe filter (or a 96 well format filter plate with 0.2 μm PTFE) and diluted as needed with ethanol.

The cannabinoid assay was run on a 1290 Infinity II Agilent HPLC system equipped with DAD, temperature-controlled column compartment, multisampler, and quaternary pump. The separation of the analytes was achieved on a Kinetex 1.7 μm EVO C18 100A 100×1.2 mm column. Full spectra were recorded from 200 to 400 nm, and absorbance at 230 nm was used to quantify cannabinoids.

Instrument control, data acquisition, and integration were achieved with OpenLAB CDS (Agilent Technologies) software, applying an identification and quantification method based on an 8-level external standards calibration curve. To confirm the analyte identity in plant material, retention time and peak purity were compared with the signal acquired on certified reference materials (CRMs).

The calibration curve used for quantification of the most common cannabinoids was obtained by analyzing serial dilutions of cannabinoid mixtures produced in house from commercially available cannabinoids CRMs. Namely, Cannabidiol (CBD), Cannabigerol (CBG), Cannabidiolic acid (CBDA) Cannabigerolic acid (CBGA), Delta-9-tetrahydrocannabinol (d9-THC), Delta-8-tetrahydrocannabinol (d8-THC), Cannabichromene (CBC), Tetrahydrocannabinolic acid (THCA), and Cannabichromenic acid (CBCA).

The content of cannabinoids is calculated in % of the dry mass of cannabis flower [% w/w]. Total CBD is calculated according to the following formula: CBD [% w/w]+CBDA*0.877 [% w/w], with the factor of 0.877 accounting for decarboxylation of the CBDA molecule. Similarly, the formulas for the other relevant major cannabinoids are: total THC=d9−THC [% w/w]+d9−THCA*0.877 [% w/w], total CBG=CBG [% w/w]+CBGA*0.877 [% w/w]; total CBC=CBC[% w/w]+CBCA*0.877 [% w/w]. The total cannabinoid content is calculated as the sum of the above total content values for single cannabinoids.

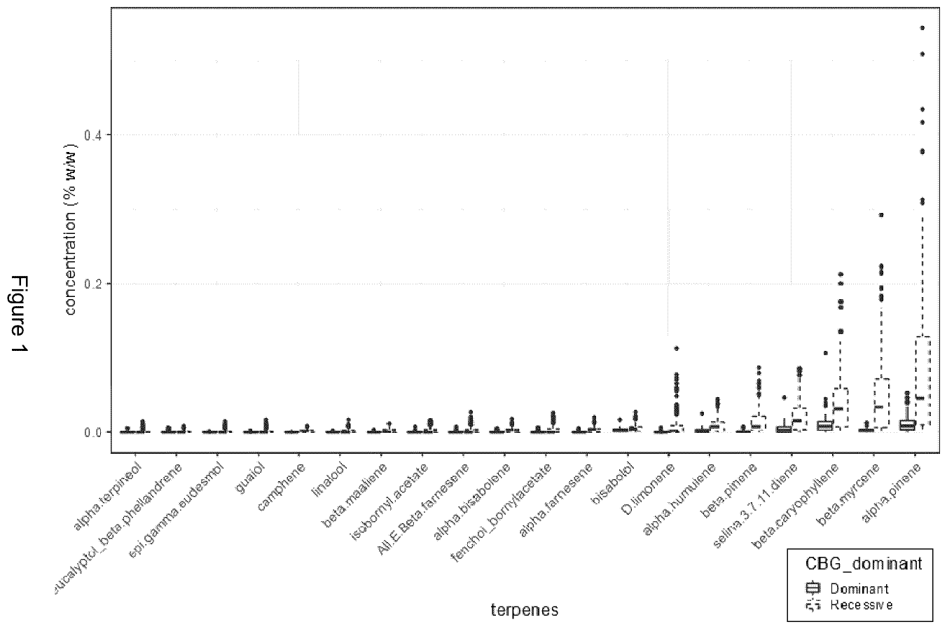

Because terpene levels had never been thoroughly reported for high CBGA plants, the inventors also characterized the terpene profiles for the segregating F2 populations (Table 1, FIG. 1). Flowers were collected at the time of harvest from the primary flowering stem in mid-October 2021, freeze-dried, and analyzed for their constituent terpene content.

The terpene profile and amount were determined on a Agilent 8890 GC system equipped with a flame ionization detector (FID) and head space sampler. One or two flower pieces were homogenously ground with a hand grinder, a 20-30 mg aliquot was weighed in a 20 ml GC glass vial. Alternatively, for exhaustive extraction of all terpenes, 500 mg of ground cannabis flower was extracted in 5 ml of ethanol (99.6%, Ph.Eur. grade) under 10 min of sonication and 30 μl of extract were added to the 20 ml GC glass vial. In the headspace sampler, samples were heated to 130° C. for 10 min before the terpene-containing gas phase was injected for analysis. Separation of terpenes was achieved on a Agilent DB HeavyWAX (30 m×250 μm×0.5 μm), using hydrogen as carrier gas.

Instrument control, data acquisition, and integration were achieved with OpenLAB CDS (Agilent Technologies) software, applying an identification and quantification method based on an 5-level external standards calibration curve. To confirm the analyte identity in plant material, retention time was compared with the signal acquired on certified reference materials (CRMs).

The calibration curve used for quantification was obtained by analyzing serial dilutions of a CRM terpene mix (SPEX Certiprep, PART #: CAN-TERP-KIT-H, Can-Terp Kit (High Level), 42 components, 1000 μg/mL (1000 ppm) in methanol containing 42 different terpenes usually present in cannabis flowers. Additional single terpenes were acquired as authentic references to identify them, using the average response factor for monoterpenes or sesquiterpenes from the 42 terpene calibration for their quantification. The terpenes were: alpha-pinene, camphene, beta-pinene, sabinene, 3-carene, beta-myrcene, alpha-phellandrene, alpha-terpinene, D-limonene, eucalyptol, beta-phellandrene, beta-ocimene (2 isomers), gamma-terpinene, p-cymene, terpinolene, fenchone, limonene-1.2-epoxide, abinene hydrate, camphor, linalool, linalyl acetate, alpha-cedrene, isopulegol, fenchol, bornyl acetate, isobornyl acetate, citral (4 isomers), beta-caryophyllene, terpinene-4-ol, menthol, pulegone, all-trans-beta-farnesene, isoborneol, alpha-humulene, alpha-terpineol, borneol, valencene, alpha-farnesene, neryl acetate, beta-bisabolene, geranyl acetate, citronellol, beta-maaliene, alpha-bisabolene, selina-3.7(11)-diene, nerol, geraniol, caryophyllene oxide, cis-nerolidol, trans-nerolidol, guaiol, epi-gamma-eudesmol, cedrol, alpha-bisabolol, alpha-eudesmol, beta-eudesmol, phytol (2 isomers).

The content of terpenes is calculated in % of the dry mass of cannabis flower (% w/w). The total terpene content is calculated as the sum of all the above listed terpenes (% w/w) (Table 1). The total monoterpene content is calculated as the sum of all the above listed monoterpenes (% w/w). The total sesquiterpene content is calculated as the sum of all the above listed sesquiterpenes (% w/w).

The inventors further devised a score for measuring adhesion based on a squeeze and release test performed by experienced members of the research team. To measure adhesion a tester would hold a sample of dried ground flower bud of approximately 1 cm in width between the index finger and the thumb and squeeze. The tester would then release pressure and evaluate the adhesion of the ground flower between their fingers on a score from 1 to 5, with 1 being non-adhesive and 5 being very adhesive. All 4 F2 populations, comprising a total of 365 individuals were scored for adhesion (Table 1). Based on scoring the inventors determined a direct correlation of low adhesion score, not adhesive, with high CBGA amount. All CBGA dominant lines were found to have adhesion scores on average of 1, being non-adhesive.

TABLE 1
Cannabinoid content, total terpene content (% w/w), and adhesion as measured in F2 populations displaying
CBGA dominant plants. The identity (ID) of the F2 population is given. CBGA dominant or recessive indicates
if the plant is determined to be CBGA dominant or recessive based on amount of CBGA (% w/w). The count
gives the number of plants used to determine the frequency of dominant and recessive plants and represents
the number of plants used to calculate mean CBGA, mean CBDA, mean THCA, and mean total terpenes. Minimum
(min) and maximum (max) CBGA, CBDA, CBCA, THCA and total terpenes (% w/w) are also provided. Average
adhesion is given as a score from 1 to 5, with 1 being non-adhesive.

| F2 | CBGA | | | | | | | | | | |
|---|---|---|---|---|---|---|---|---|---|---|---|
| Population | dominant/ | | Population | | Mean | Min | Max | Mean | Min | Max | Mean |
| ID | recessive | Count | sum | Frequency | CBGA | CBGA | CBGA | CBDA | CBDA | CBDA | CBCA |
| 21 002 016 | Dominant | 13 | 75 | 0.173 | 5.11 | 2.60 | 7.93 | 0.16 | 0.07 | 0.35 | 0.27 |
| 21 002 029 | Dominant | 16 | 91 | 0.176 | 5.71 | 4.10 | 7.10 | 0.23 | 0.07 | 0.71 | 0.31 |
| 21 002 036 | Dominant | 31 | 112 | 0.277 | 5.35 | 2.11 | 9.42 | 0.25 | 0.05 | 0.92 | 0.20 |
| 21 002 041 | Dominant | 18 | 87 | 0.207 | 6.02 | 2.34 | 9.75 | 0.29 | 0.09 | 0.45 | 0.39 |
| 21 002 016 | Recessive | 62 | 75 | 0.827 | 0.43 | 0.12 | 1.18 | 8.69 | 4.24 | 13.93 | 1.11 |
| 21 002 029 | Recessive | 75 | 91 | 0.824 | 0.31 | 0.06 | 1.40 | 7.21 | 0.16 | 14.33 | 1.46 |
| 21 002 036 | Recessive | 81 | 112 | 0.723 | 0.34 | 0.00 | 1.91 | 7.71 | 2.75 | 14.09 | 1.17 |
| 21 002 041 | Recessive | 69 | 87 | 0.783 | 0.36 | 0.00 | 1.08 | 9.29 | 5.69 | 15.58 | 1.88 |

| F2 | CBGA | | | | | | Min | Max | Mean | Average |
|---|---|---|---|---|---|---|---|---|---|---|
| Population | dominant/ | Min | Max | Mean | Min | Max | Total | Total | Total | Adhesion |
| ID | recessive | CBCA | CBCA | THCA | THCA | THCA | terpenes | terpenes | terpenes | Score |
| 21 002 016 | Dominant | 0.16 | 0.48 | 0.06 | 0.00 | 0.08 | 0.00 | 0.04 | 0.02 | 1 |
| 21 002 029 | Dominant | 0.19 | 0.45 | 0.07 | 0.05 | 0.10 | 0.00 | 0.03 | 0.01 | 1 |
| 21 002 036 | Dominant | 0.06 | 0.89 | 0.03 | 0.00 | 0.09 | 0.02 | 0.15 | 0.05 | 1 |
| 21 002 041 | Dominant | 0.11 | 1.25 | 0.08 | 0.04 | 0.19 | 0.00 | 0.25 | 0.06 | 1 |
| 21 002 016 | Recessive | 0.35 | 2.47 | 0.30 | 0.16 | 0.48 | 0.00 | 0.71 | 0.21 | 4 |
| 21 002 029 | Recessive | 0.23 | 2.92 | 0.24 | 0.00 | 0.48 | 0.00 | 0.12 | 0.04 | 4 |
| 21 002 036 | Recessive | 0.26 | 3.09 | 0.23 | 0.09 | 0.48 | 0.02 | 1.12 | 0.37 | 4 |
| 21 002 041 | Recessive | 0.64 | 3.74 | 0.31 | 0.19 | 0.51 | 0.00 | 0.92 | 0.39 | 4 |

All F2 populations used in this study displayed an occurrence of CBGA dominance with a frequency of between 17-28%, indicating that the trait is recessive and likely monogenic. CBGA levels above 4 (% w/w) were used as an indicator of CBGA dominance.

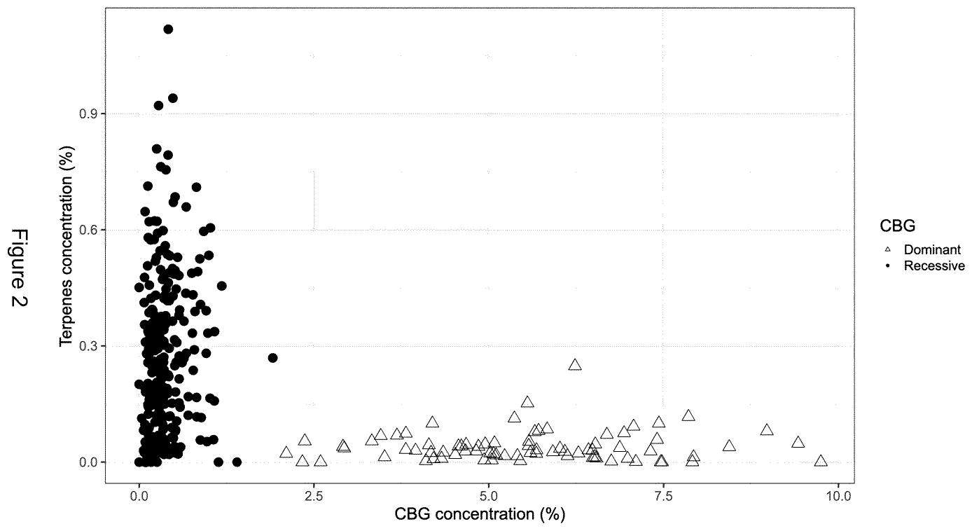

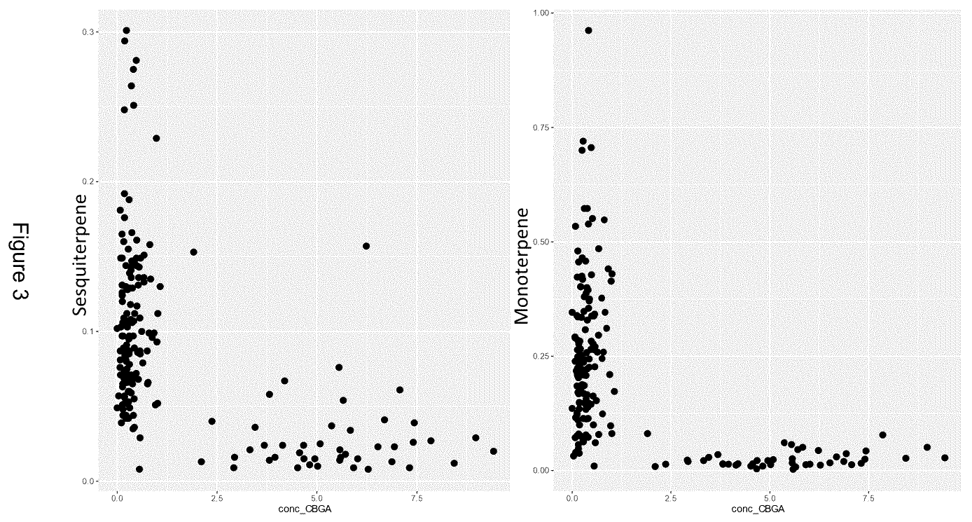

The evaluation of the cannabinoid levels of the F2 populations showed that plants with average high CBGA levels >2 (% w/w) also had much lower THCA, CBDA, and CBCA (Table 1). When evaluating terpene levels in the F2 populations, the inventors surprisingly found that plants segregating for CBGA levels had almost no terpenes as compared to the plants of the F2 population with low CBGA levels (Table 1, FIG. 2). The inventors evaluated monoterpene and sesquiterpene levels as well and found that both mono- and sesqui-terpene levels were significantly lower on average in plants with high CBGA levels (FIG. 3). To their knowledge, this inverse correlation has not previously been described and has significant industrial application. Further, likely as a result of the low cannabinoid and terpene levels, the CBGA dominant lines displayed a much lower adhesion profile.

### Example 2

### Genome-Wide Association Studies (GWAS) of CBGA Dominant Cannabis

In order to identify markers that associate with low terpene/cannabinoid levels, the inventors reasoned that CBGA levels could be used as a proxy for terpene and cannabinoid levels as there is an excellent correlation between low terpene/cannabinoid and high CBGA levels.

All F2 plants described in Table 1 were sequenced. DNA was extracted from about 70 mg of leaf discs from all the plants evaluated using an adapted kit with “sbeadex” magnetic beads by LGC Genomics, which was automated on a KingFisher Flex with 96 Deep-Well Head by Thermo Fisher Scientific.

The extracted DNA served as a template for the subsequent library preparation for sequencing. The library pools were prepared according to the manufacturer's instructions (AgriSeq™ HTS Library Kit-96 sample procedure from Thermo Fisher Scientific). Targeted sequencing of a custom SNP marker panel based on the Cannabis Sativa CS10 reference genome (NCBI GenBank assembly accession number GCA_900626175.2 as updated in April 2020 and accessed in February 2022) was carried out on the Ion Torrent system by Thermo Fisher Scientific. The primers for the SNPs identified are provided in Table 4 below. The library pool was loaded onto Ion 550 chips with Ion Chef and sequenced with Ion GeneStudio S5 Plus according to the manufacturer's instructions (Ion 550™ Kit from Thermo Fisher Scientific).

From a population of 4 combined F2 populations with a total of 365 individuals, a genome-wide association study (GWAS) was performed to detect significant associations between genotypic information derived from targeted resequencing of the custom SNP marker panel described above and CBGA amount (% w/w) measured in flowers of the F2 populations.

The genotypic matrix was filtered for <30% missing values and <1% minor allele frequency before the association. The final number of SNPs used for the GWAS was 3485, number of individual plants: 329. GWAS was performed using GAPIT3 using four models (GLM, MLM, Blink and FarmCPU). SNPs were considered significant if surpassing the Bonferroni-corrected threshold (−log10/0.05/number of markers) of 4.84 LOD. The inventors chose to use the BLINK model because it performed the best by their evaluation.

Specifically, the inventors identified a quantitative trait locus (QTL1) associated with high CBGA on chromosome NC_044378.1 defined by SNP “common_4934” and “common_4897”, having positions, with respect to the CS10 reference genome, respectively at positions 55368831-61932481 (Table 2). The SNP most significantly associated with QTL1 is “common_4934”, with an LOD score of 29.7, for which the allelic variants listed can be used to be used to distinguish CBGA dominant plants from non-dominant plants (Table 2). In QTL1, when SNP “common_4934” is Allele_1, homozygous AA, this indicates a plant with a significantly increased CBGA levels as compared to the alternative allele, Allele_3. Interestingly, the heterozygous state is associated with low CBGA levels rather than intermediate CBGA levels, as might be expected if the causative polymorphism impacted the activity of an enzyme in cannabinoid biosynthesis.

The inventors examined the SNP most significantly associated with variance in CBGA amount, “common_4934”, for its impact on terpene levels. They found that the allelic variation of “common_4934” is also strongly associated with reduced terpene amount. The presence of the allelic variant AA at “common_4934” is associated with an almost 10 fold decrease in total terpene amount as compared to the alternative variant. The heterozygous state of the allele showed terpene levels that were similar to the total terpene levels found in the alternative variant.

Upon examination of all SNPs identified in the GWA, only “common_4934” showed an association with variation in terpene amount and was thus focused on by the inventors. Importantly not all of the SNPs identified as being significantly associated with CBGA dominance were associated with variation in terpene level. “Common_4934” fulfilled both being a marker for CBGA dominance (and decreased cannabinoid levels) and for decreased terpene level. The reference sequence for each of the SNPs identified is given in Table 3 with reference to the CS10 genome. In Table 4, PCR primers designed to amplify each of the regions containing these SNPs, with reference to the CS10 genome, are provided in order for the allelic variant to be determined.

The inventors were mindful of U.S. Patent Application Publication Number 2022/0228159 A1, in which a polymorphism in a THCA synthase was found to be associated with the CBGA dominant phenotype. A BLAST search of the CS10 reference genome assembly using sequences specific to the THCAS G1064A polymorphism (SEQ ID NO:1-AAAAACTGATTGCAAAGAATTTAGCTGGATTGATACAACCATCTTCTACA[A/G]TGGTGTT GTAAATTTTAACACTGCTAATTTTAAAAAGGAAATTTTGCTTG) found this polymorphism to be at position 25823020 with respect to the CS10 reference genome assembly. The inventors tested the hypothesis that the representation of the positions of “common_4934” and the THCAS G1064A polymorphism on the CS10 reference genome assembly may be showing them at opposite sides of the chromosome when in fact they might be closer to each other. Using the reference sequence for the THCAS G1064A polymorphism (SEQ ID NO:1) and for “common_4934” (SEQ ID NO:2) the inventors used BLAST to identify their positions on five previously assembled Cannabis sativa reference genomes.

The BLAST search found that the THCAS G1064A polymorphism and “common_4934” are located on the same genomic scaffold and are within 2.3-4.5 Mb of each other in all reference genomes searched in our collection. It may be that the CS10 genome is misassembled in this region.

TABLE 2
SNPs associated with CBGA in the F2 populations. CBGA dominance is predicted by the occurrence of
the indicative allele (marked with *). Decreased terpene levels is predicted by the occurrence of
the indicative allele (marker with +). The positions and chromosome of the SNPs are provided
with reference to the CS10 reference genome as described herein. The LOD score is provided for the
BLINK model association for CBGA. “CBGA Mean 1” denotes the average phenotypic value associated
with Allele 1 based on mean CBGA amount (% w/w), “CBGA Mean 2” denotes the average phenotypic
value associated with Allele 2 based on mean CBGA amount (% w/w) and “CBGA Mean 3” denotes
the average phenotypic value associated with Allele 3 based on mean CBGA amount (% w/w). “Total
terpenes Mean 1”, “Total terpenes Mean 2”, and “Total terpenes Mean 3” denote the
mean phenotypic value of total terpene amount (% w/w) from Allele 1, Allele 2, and Allele 3, respectively.
Count 1, Count 2, and Count 3 denote the number of plants that contributed to the average phenotypic
value of Mean 1, Mean 2, and Mean 3 for both CBGA and terpenes, respectively. The SNP position on
the chromosome is provided with reference to the CS10 reference genome.

| | | | | Allele | Allele | Allele | CBGA | CBGA |
|---|---|---|---|---|---|---|---|---|
| SNP | Chromosome | Position | LOD | 1 | 2 | 3 | Mean 1 | Mean 2 |
| common_4934 | NC_044378.1 | 61932481 | 29.6941121 | AA*+ | AG | GG | 5.12282027 | 0.559683851 |
| common_4931 | NC_044378.1 | 61651644 | 13.7246782 | CC | CG | GG* | 0.28808 | 0.414437069 |
| common_4897 | NC_044378.1 | 55368831 | 10.5838253 | AA | AG | GG | 0.3131 | 0.980755 |
| common_5468 | NC_044379.1 | 58503008 | 7.48314636 | AA | AC | CC | 1.459273077 | 1.439996341 |
| GBScompat_{—} | NC_044379.1 | 24979664 | 6.96518314 | AA | AG | GG | 1.161771642 | 1.353139181 |
| common_995 | | | | | | | | |
| common_1244 | NC_044372.1 | 7351543 | 6.56131894 | AG | GG | NA | 0.2972 | 1.466692675 |
| common_3444 | NC_044375.1 | 92776712 | 4.87906649 | AA | AG | NA | 1.571771898 | 1.064714706 |

| | | Total | Total | Total | | | |
|---|---|---|---|---|---|---|---|
| | CBGA | terpenes | terpenes | terpenes | Count | Count | Count |
| SNP | Mean 3 | Mean 1 | Mean 2 | Mean 3 | 1 | 2 | 3 |
| common_4934 | 0.1756625 | 0.05544595 | 0.26921118 | 0.3313125 | 74 | 161 | 32 |
| common_4931 | 2.847765217 | 0.2854 | 0.26957759 | 0.20415217 | 5 | 116 | 138 |
| common_4897 | 0.452712 | 0.376 | 0.29955 | 0.275415 | 1 | 20 | 200 |
| common_5468 | 1.687401316 | 0.19776923 | 0.21773171 | 0.25538158 | 52 | 164 | 76 |
| GBScompat_{—} | 2.706545455 | 0.24771642 | 0.21377778 | 0.22170455 | 67 | 171 | 144 |
| common_995 | | | | | | | |
| common_1244 | 3.121484615 | 0.442 | 0.22099363 | 0.20846154 | 2 | 314 | 13 |
| common_3444 | 1.659495238 | 0.22854745 | 0.18167647 | 0.19938095 | 274 | 34 | 21 |

TABLE 3
Detailed information of each of the SNPs associated with CBGA dominance in
Cannabis spp. as provided in Table 2. The “context sequence” is given with
the SNP given in brackets. All of the sequences and alleles are provided
with reference to the plus strand.

| SNP | Context Sequence |
|---|---|
| common_4934 | CCAAGATCAAGATCTCTATAGTTCACATAAGCTTCCCTTGGAAATCTAGACACATATGGA |
| | CCCATGTAATCGTAAAGCCTTTGAATCCACTTCACGTGTTTAGCTGAACTTTTTGGGCTT |
| | CCATCGAACCAAGCCGTAAGGTATTGAATCATGAAAATGACTCCTTTTCTGTGTGGAAA |
| | TGGGATTTGGGTCTCTGGAATT[C/T]TGCTCATCATTCCCCCATATGGTGTCCAAATCGT |
| | TAAAGGAGTATCTTCTTCCAAAAACCTTTTCCAAACCCCTTCTAGTGTTGTTTCTGGTATT |
| | GGGTCAGTCACAAAATCAGATTTTGCTTTGAAGTAAACTTTTGATGTCGTTTTCCCTTGG |
| | AGCAAAATCTCAGGTGGGGTTCCACTTGGGTATGTTCCAGAGATGT |
| | (SEQ ID NO: 2) |
| common_4931 | TTTTGCTTTGCTTATGAGAAAATAATACATTAATACCTTGGTAACAAATGTACCATTACGA |
| | GCCAGAAAATGAGTAACCAACTTAAGGGCATCGATGACCAAACCATTCTGAACAGTGGC |
| | TTCCATATTCCAAGCACCGCCCATTTTTGGGGCACCATCGTGCAGAACCAAATCGAAGG |
| | CAGAGAACTCATACTTCCTCAT[C/G]ACCCTTTTGATCTCCGACTTGCATTGGGGTTTGG |
| | TGATGTCCTGCTTAATAAAAATGGCGCCTGGAATTGGATTAACCCAATTCAAATCCACAC |
| | CCACAATAAGGCTTCCACGTGGCACCCGTAGAGTGGCTACCTGCATCCAACTGCCTGG |
| | AGCAGCACAGAGGTCGAGAACAGCCCCAGTTTTGTGAAACAATCCATAC |
| | (SEQ ID NO: 3) |
| common_4897 | TACATGGAAAGTATATGTTGACGGAGCGTCAAATGAAAATGGATCGGGGGCCGAAATA |
| | GCAATGATCTCTCCTAACAGGCTAAGACTCCAGTCTGCCCTACAGTTCACATTTCTGGC |
| | TTCTAACAATGAGGCTGAGTACAAAGCCCTGGTAGCAAAATTAAAATTGGCTAAGGTTG |
| | TAGGGGCCAACAGGGTAGAAATGTA[T/C]AGTGATTCGCAACTGGTGGTCAACCAGGTA |
| | TTAGGGGAATATCAGACTTGGGGAGAAAAGATGGCTGCTTACATATCGGTTGTTAGGGA |
| | ATTGCTGCAAGAGTTTAAGGAATACAAAGTGGAACGAATTCCACGAGAAAGAAATGCCC |
| | ATGCAAACTGTCTGGCTAAGCTAGCCTTAGATAGCGAGATAGAGAAGCTGGGG |
| | (SEQ ID NO: 4) |
| common_5468 | ATGGGAATCTCCTCCTAGGCCTTTCCATGAAATCCCACACTACTACCATGAAAACATGT |
| | CAATGGAGTCTCTTTGCAGACTTCATGGATGGGGAATTCGGGAATCTCCAAGGCGAGT |
| | CTATGATGCGGTTTTATTCAGTAATGAAGTGGACATCCTTACAATCAGATGGAATGAATT |
| | GTATCCATATGTGACACAGTTTGT[T/G]CTTCTTGAGTCAAACTCAACTTTCACTGGTTTG |
| | CCAAAGCCTTTAATTTTCTCTGGAAACAGGGACAAATTTAAGTTTATTGAGCCTCGGCTT |
| | ACTTATGGTATGTTGGGGGGAAGGTTTCATAGAGGCGAAAACCCTTTTGTGGAGGAGG |
| | CATATCAGAGAGTAGCATTGGATCAGCTTCTCAGAATAGCAGGCATAGAA |
| | (SEQ ID NO: 5) |
| GBScompat_ | ATCTACCCTTTGAGAAAGTGAAACGGAAGCACGTGAAGCTTCTGATACTACAGTTGAAA |
| common_995 | CATGATGATCAGTTGGAGATGGAGCCTCACTCTGAGAAAAAGCGGGATGGCTAGCATC |
| | TAATTGACCAGATCGTGGCCGTTGAACTGGTCCCTCGAGTGGAAAGCCACCCCCAGCA |
| | GAAATAGAAGCTAGACGAGTAGCCTC[G/A]TTTAAACTATGAAGAGTGTTTATAAGCCTC |
| | AAAAGTATTCCATTCTTTGCAGCTATACGACAAAAATCATTTCTAGGTGTAGAGCGCTGA |
| | AGCTTAAAGACTTGCCACATGCCATCAATAGCTAGATGAACCATTTCCCTGTATGTGAA |
| | GATACTAATGAGCAAATTTCAAACTGAAACTATAAAGTATTGAACAACGTAC |
| | (SEQ ID NO: 6) |
| common_1244 | TATAATGATGGAAGCTTACCAGGGTTGATTATAAAAGTCAGAACGCCGCTCCTTCTCTG |
| | CATTTCGACTGGCATCTCCAGCAACAAGCTTTAAATCTTTGCTCTGAGAAGCTATCAGA |
| | GTGTTAATAAACTCTGCTGGAGACTGACTGAAACTAAGAAAGAAAGCCCGCCGCCTCCT |
| | ATGTTCATGAATCTTCTTTATAGA[T/C]GCAGATATCAGTTCATCGCAAGCATCAATTTCC |
| | TTATTATTCTCAGTACGTGCCAAGAATGAAGACATTTCTTTATCCAGAGAAAGAGGCACA |
| | TCAACCAACACATCATAGCAATTAGTTCCAACTGGACAAGTTCCCGAAAGCTTAATCTTA |
| | TGTTCCAGATGTATGGGTTGTGGTGGAGTTAAATGTGTGCTAACTTT |
| | (SEQ ID NO: 7) |
| common_3444 | TAGTTAATTTGTTTTCCAGTTACTACTAAGCTGCTTCAGCCTCCAATAACTTTCTTTCGGC |
| | CAAAGCATCCACAGCTTTTACTTTCTCAGGTACAAAATCTACAAACATTTCAAGACATTA |
| | CATCAGAGAGAACAATAATGGCATAATAGTTGAAAAAAAATGTTTAAAATCGTCTATGAA |
| | AAGTATAAGGTACCTGAAAG[G/A]AAATCGTATCTCTTCCGTTTGCTCACAACTGATGCT |
| | GAAAAAACAAACTTGTACTTAAGTACTGCTAGCATATAAATGATAAAAATTGTTTTGTAAT |
| | TAATTGTAAGAAACAATTAGAGTTAAGAGTACCAAGTTGCCTAACCCCTCTCTACAGTAT |
| | AAGCTGAGCATAAATAACTGGTGATCCTCTCAAGAGAGAGAATA |
| | (SEQ ID NO: 8) |

TABLE 4
Targeted sequencing primers (5' to 3') for the SNPs identified in Table 2, as described
in Example 2.

| | Forward | Reverse | Forward | Reverse | Forward | Reverse |
|---|---|---|---|---|---|---|
| SNP | Primer 1 | Primer 1 | Primer 2 | Primer 2 | Primer 3 | Primer 3 |
| common_4934 | TTGGGCTTCC | ACATACCCAA | TTTGGGCTTC | CATACCCAAG | TTTGGGCTTC | ACATACCCAA |
| | ATCGAACCAA | GTGGAACCCC | CATCGAACCA | TGGAACCCCA | CATCGAACCA | GTGGAACCCC |
| | (SEQ ID | (SEQ ID | (SEQ ID | (SEQ ID | (SEQ ID | (SEQ ID |
| | NO: 9) | NO: 10) | NO: 11) | NO: 12) | NO: 11) | NO: 10) |
| common_4931 | GGCATCGATG | AAACTGGGGC | GGCATCGATG | AAAACTGGGG | GGCATCGATG | AAACTGGGGC |
| | ACCAAACCAT | TGTTCTCGAC | ACCAAACCAT | CTGTTCTCGA | ACCAAACCATT | TGTTCTCGAC |
| | NO: 13) | NO: 14) | NO: 13) | NO: 15) | NO: 16) | NO: 14) |
| common_4897 | GCTGAGTACA | CCCCAGCTTC | GCTGAGTACAA | CCCCAGCTTC | GCTGAGTACA | CCCAGCTTCT |
| | AAGCCCTGGT | TCTATCTCGC | AGCCCTGGTA | TCTATCTCGC | AAGCCCTGGT | CTATCTCGCT |
| | (SEQ ID | (SEQ ID | (SEQ ID | (SEQ ID | (SEQ ID | (SEQ ID |
| | NO: 17) | NO: 18) | NO: 19) | NO: 18) | NO: 17) | NO: 20) |
| common_5468 | GCAGACTTCA | ATGCCTCCTC | TGCAGACTTC | ATGCCTCCTC | TGCAGACTTC | GCCTCCTCCA |
| | TGGATGGGGA | CACAAAAGGG | ATGGATGGGG | CACAAAAGGG | ATGGATGGGG | CAAAAGGGTT |
| | (SEQ ID | (SEQ ID | (SEQ ID | (SEQ ID | (SEQ ID | (SEQ ID |
| | NO: 21) | NO: 22) | NO: 23) | NO: 22) | NO: 23) | NO: 24) |
| GBScompat_ | AGAAAGTGAAA | GCTTCAGCGC | GAGAAAGTGAA | GCTTCAGCGC | GAAAGTGAAA | GCTTCAGCGC |
| common_995 | CGGAAGCACG | TCTACACCTA | ACGGAAGCACG | TCTACACCTA | CGGAAGCACG | TCTACACCTA |
| | (SEQ ID | (SEQ ID | (SEQ ID | (SEQ ID | (SEQ ID | (SEQ ID |
| | NO: 25) | NO: 26) | NO: 27) | NO: 26) | NO: 28) | NO: 26) |
| common_1244 | TGGAAGCTTA | TGTGTTGGTT | ATGGAAGCTTA | TGTGTTGGTTG | TGGAAGCTTA | TGTGTTGGTTG |
| | CCAGGGTTGA | GATGTGCCTC | CCAGGGTTGA | ATGTGCCTCT | CCAGGGTTGA | ATGTGCCTCT |
| | (SEQ ID | (SEQ ID | (SEQ ID | (SEQ ID | (SEQ ID | (SEQ ID |
| | NO: 29) | NO: 30) | NO: 31) | NO: 32) | NO: 29) | NO: 32) |
| common_3444 | TTCGGCCAAA | TGTAGAGAGG | GGCCAAAGC | ACTGTAGAGAG | TTCGGCCAAA | TGTAGAGAGG |
| | GCATCCACAG | GGTTAGGCAAC | ATCCACAGCT | GGGTTAGGCA | GCATCCACA | GGTTAGGCAAC |
| | (SEQ ID | (SEQ ID | (SEQ ID | (SEQ ID | (SEQ ID | (SEQ ID |
| | NO: 33) | NO: 34) | NO: 35) | NO: 36) | NO: 37) | NO: 34) |

The inventors next sought to confirm the use of “common_4934” in predicting the low terpene phenotype as well as comparing it to the previously described G1064A polymorphism in a gene encoding a defunct THCAS synthase.

The inventors used a Kompetitive allele-specific PCR (KASP) marker to test the polymorphism THCAS G1064A in an inactive THCA synthase that had previously been shown to be associated with the high CBGA trait. For the KASP-assay, DNA was extracted from 52 plants composed of selections from the F2 populations described in Table 1, as well as parents and grandparents of these populations, and other distinct genotypes known to be CBGA dominant or recessive, using the QuickExtract Plant DNA Extraction Solution from LGC Genomics. The extraction was performed following the manufacturer's guidelines. The primers incorporate the targeted SNP, which enables bi-allelic scoring of the SNP of interest. KASP primers for the assay were designed at LGC Genomics. The KASP Assay mix contains three assay-specific non-labeled oligos: two allele-specific forward primers and one common reverse primer. For KASP, primers were designed to detect the single nucleotide polymorphism THCAS G1064A, named as KASP3. The primers used were:

Ref_G Fwd-
(SEQ ID NO: 38)
AATTAGCAGTGTTAAAATTTACAACACCAC;
Alt_A Fwd-
(SEQ ID NO: 39)
AAAATTAGCAGTGTTAAAATTTACAACACCAT;
and
Common_Rv-
(SEQ ID NO: 40)
ATTTAGCTGGATTGATACAACCATCTTCTA.

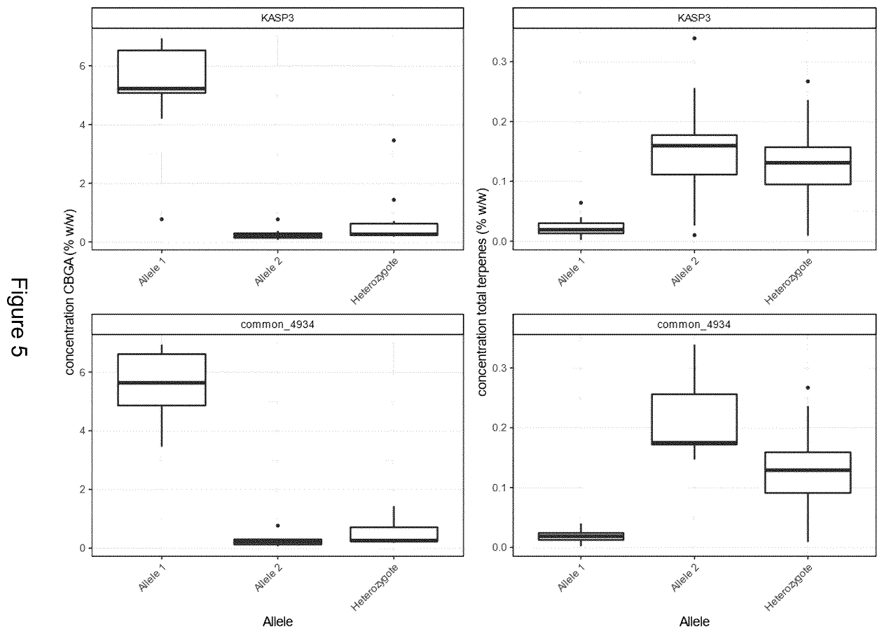

The allele-specific primers each harbor a unique tail sequence that corresponds with a universal FRET (fluorescence resonant energy transfer) cassette; one labelled with FAM™ dye and the other with HEX™ dye. The KASP Master mix contains the universal FRET cassettes, ROX™ passive reference dye, taq polymerase, free nucleotides, and MgCl₂ in an optimized buffer solution. During thermal cycling, the relevant allele-specific primer binds to the template and elongates, thus attaching the tail sequence to the newly synthesized strand. The complement of the allele-specific tail sequence is then generated during subsequent rounds of PCR, enabling the FRET cassette to bind to the DNA. The FRET cassette is no longer quenched and emits fluorescence. Bi-allelic discrimination is achieved through the competitive binding of the two allele-specific forward primers. If the genotype at a given SNP is homozygous, only one of the two possible fluorescent signals will be generated. If the genotype is heterozygous, a mixed fluorescent signal will be generated. As the fluorescent signals are generated at the end of the thermal cycling and as those signals are clustered, three allelic groups can be differentiated: homozygous for Allele 1, heterozygous, and homozygous for Allele 2. All DNA analysed for the KASP assay described here also underwent targeted resequencing on the custom SNP marker panel as described above (Table 4). This allowed the determination of the allelic variants of marker “common_4934” homozygous for Allele 1, heterozygous, and homozygous for Allele 2 to be made, with reference to Table 2. The KASP results are presented in relation to the concentration of CBGA (% w/w) and total terpene (% w/w) in FIG. 5. The inventors found that “common_4934” could perfectly predict plants with low terpene amount and those with high CBGA amount, and thus low THCA, CBDA, and CBCA amount with reference to Table 2 and FIG. 5. KASP3 however was found by the inventors to be 95% predictive, improperly identifying a low CBGA plant as one that was a high CBGA genotype and identifying a CBGA dominant plant as heterozygote. The inventors convincingly found that “Common_4934” outperformed the KASP3 marker for both determining CBGA, cannabinoid and terpene amounts.

This indicates that Common_4934 is highly linked to the low terpene trait and is more closely linked than the previously known polymorphism, THCAS G1064A, is to the high CBGA trait.

Interestingly the inventors found a surprising result that will be of benefit in identifying and developing varieties with modulated amounts of CBGA and terpene. As noted above, Allele 1 of SNP “common_4934” (AA) can predict low terpene levels where CBGA levels are above 2 (% w/w). This marker can also be useful in identifying plants heterozygous for “common_4934” (AG) where terpene levels are not practically eliminated but that also have increased CBGA levels with a mean of 0.55 (% w/w) in the ranges from 0.15-2 (% w/w). This is compared to plants that are homozygous (GG) i.e., carrying Allele 3 where CBGA levels rarely are higher than 0.08 (% w/w).

The marker common_4934 allowed the inventors to rapidly screen F2 populations for CBGA dominance/low terpene amount, allowing parallel determination of other agriculturally important traits using markers specific to those traits and traditional trait-based characterization of mature plants. This greatly facilitated identification of those additional traits coupled to CBGA dominance.

### REFERENCES

Garfinkel, A. R.; Otten, M.; Crawford, S. SNP in Potentially Defunct Tetrahydrocannabinolic Acid Synthase Is a Marker for Cannabigerolic Acid Dominance in Cannabis sativa L. Genes 2021, 12, 228.
Luo, X.; Reiter, M. A.; d'Espaux, L.; Wong, J.; Denby, C. M.; Lechner, A.; Zhang, Y.; Grzybowski, A. T.; Harth, S.; Weiyin Lin, Lee, H.; Yu, C.; Shin, J.; Deng, K.; Benites, V. T.; Wang, G.; Baidoo, E. E. K.; Chen, Y.; Dev, I.; Petzold, C. J.; Keasling, J. D. Complete biosynthesis of cannabinoids and their unnatural analogues in yeast. Nature 2019, 567, 123-126.

1. A method for identifying a Cannabis spp. plant having a high cannabigerolic acid (CBGA) trait, the method comprising the steps of:
(i) genotyping at least one plant with respect to at least one high CBGA QTL by detecting one or more polymorphisms associated with the high CBGA trait as defined in Table 2; and
(ii) identifying the plant as having the at least one high CBGA QTL based on the genotype at the polymorphism, wherein the at least one high CBGA QTL is associated with the high CBGA trait.

2. The method of claim 1, wherein the polymorphism is “common_4934”, as defined in Table 2.

3. The method of claim 2, wherein the polymorphism “common_4934” has the state of Allele 1 or Allele 2, as defined in Table 2.

4. The method of claim 1, wherein the genotyping is performed by PCR-based detection using molecular markers, sequencing of PCR products containing the one or more polymorphisms, targeted resequencing, whole genome sequencing, or restriction-based methods, for detecting the one or more polymorphisms.

5. The method of claim 4, wherein the molecular markers are for detecting polymorphisms at regular intervals within the at least one high CBGA QTL such that recombination can be excluded or can be quantified to estimate linkage disequilibrium between a particular polymorphism and the high CBGA trait.

6. (canceled)

7. The method of claim 4, wherein the molecular markers are designed based on a context sequence for the polymorphism in Table 3 or are selected from the primer pairs as defined in Table 4.

8. The method of claim 1, wherein the high CBGA QTL is a quantitative trait locus having a sequence that corresponds to nucleotides 55368831-61932481 of NC_044378.1 with reference to the CS10 genome and is defined by one or more polymorphism selected from the group consisting of “common_4934”, “common 4931” and “common_4897” associated with high CBGA and low terpene/cannabinoid levels as defined in Table 2, or a genetic marker linked to the QTL.

9. A method of producing a Cannabis spp. plant having a high CBGA trait, the method comprising the steps of:
(i) providing a donor parent plant having in its genome at least one high CBGA QTL characterized by one or more polymorphisms associated with the high CBGA trait as defined Table 2;
(ii) crossing the donor parent plant having the at least one high CBGA QTL with at least one recipient parent plant to obtain a progeny population of cannabis spp. plants;
(iii) screening the progeny population of cannabis spp. plants for the presence of the at least one high CBGA QTL; and
(iv) selecting one or more progeny plants having the at least one high CBGA QTL, wherein the mature plant displays the high CBGA trait.

10. The method of claim 9, further comprising:
(v) crossing the one or more progeny plants with the donor recipient plant; or
(vi) selfing the one or more progeny plants.

11. The method of claim 9, wherein the screening comprises genotyping at least one plant from the progeny population with respect to the at least one high CBGA QTL by detecting one or more polymorphisms associated with the high CBGA trait as defined Table 2, and optionally wherein the method further comprises a step of genotyping the donor parent plant with respect to the at least one high CBGA QTL by detecting one or more polymorphisms associated with the high CBGA trait as defined Table 2.

12. (canceled)

13. The method of claim 11, wherein the genotyping is performed by PCR-based detection using molecular markers, sequencing of PCR products containing the one or more polymorphisms, targeted resequencing, whole genome sequencing, or restriction-based methods, for detecting the one or more polymorphisms.

14. The method of claim 13, wherein the molecular markers are for detecting polymorphisms at regular intervals within the at least one high CBGA QTL such that recombination can be excluded or such that recombination can be quantified to estimate linkage disequilibrium between a particular polymorphism and the high CBGA trait, optionally wherein the molecular markers are designed based on a context sequence for the polymorphism in Table 3 or are selected from the primer pairs as defined in Table 4.

15. (canceled)

16. The method of claim 9, wherein the polymorphism is “common_4934”, as defined in Table 2, optionally having the state of Allele 1 or Allele 2, as defined in Table 2.

17. (canceled)

18. The method of claim 9, wherein the high CBGA QTL is a quantitative trait locus having a sequence that corresponds to nucleotides 55368831-161932481 of NC_044378.1 with reference to the CS10 genome and is defined by one or more polymorphism selected from the group consisting of “common_4934”, “common 4931” and “common_4897” associated with high CBGA and low terpene/cannabinoid levels as defined in Table 2, or a genetic marker linked to the QTL.

19. A method of producing a Cannabis spp. plant that has a high CBGA trait, the method comprising introducing at least one high CBGA QTL characterized by one or more polymorphisms associated with the high CBGA trait as defined in Table 2 into a Cannabis spp. plant, wherein said high CBGA QTL is associated with the high CBGA trait in the plant.

20. The method of claim 19, wherein introducing the at least one high CBGA QTL comprises crossing a donor parent plant having the at least one high CBGA QTL characterized by one or more polymorphisms associated with the high CBGA trait with a recipient parent plant, or genetically modifying the Cannabis spp. plant.

21. (canceled)

22. The method of claim 19, wherein the high CBGA QTL is a quantitative trait locus having a sequence that corresponds to nucleotides 55368831-61932481 of NC_044378.1 with reference to the CS10 genome and is defined by one or more polymorphism selected from the group consisting of “common_4934”, “common 4931” and “common_4897” associated with high CBGA and low terpene/cannabinoid levels as defined in Table 2, or a genetic marker linked to the QTL.

23. (canceled)

24. (canceled)

25. (canceled)

26. A Cannabis spp. plant comprising at least one high CBGA QTL characterized by one or more polymorphisms associated with a high CBGA trait as defined in Table 2, wherein said high CBGA QTL is associated with the high CBGA trait in the plant.

27. (canceled)

28. (canceled)

29. A plant extract obtained from a Cannabis spp. plant of claim 26.

30. The plant extract of claim 29, wherein the plant extract has a cannabinoid and terpene profile with minimum and maximum cannabinoid and terpene ranges as set out for a CBGA dominant plant in Table 1.


SEQUENCE LISTING (40 sequences)

SEQ ID NO: 1 - DNA, 101 bp, Cannabis sativa
  source 1..101: mol_type = genomic DNA
  variation 51: allele = N is A or G
aaaaactgattgcaaagaatttagctggattgatacaaccatcttctacantggtgttgtaaattttaacactgctaattttaaaaaggaaattttgcttg

SEQ ID NO: 2 - DNA, 403 bp, Cannabis sativa
  source 1..403: mol_type = genomic DNA
  variation 202: allele = N is C or T
ccaagatcaagatctctatagttcacataagcttcccttggaaatctagacacatatggacccatgtaatcgtaaagcctttgaatccacttcacgtgtttagctgaactttttgggcttccatcgaaccaagccgtaaggtattgaatcatgaaaatgactccttttctgtgtggaaatgggatttgggtctctggaattntgctcatcattcccccatatggtgtccaaatcgttaaaggagtatcttcttccaaaaaccttttccaaaccccttctagtgttgtttctggtattgggtcagtcacaaaatcagattttgctttgaagtaaacttttgatgtcgttttcccttggagcaaaatctcaggtggggttccacttgggtatgttccagagatgt

SEQ ID NO: 3 - DNA, 403 bp, Cannabis sativa
  source 1..403: mol_type = genomic DNA
  variation 202: allele = N is C or G
ttttgctttgcttatgagaaaataatacattaataccttggtaacaaatgtaccattacgagccagaaaatgagtaaccaacttaagggcatcgatgaccaaaccattctgaacagtggcttccatattccaagcaccgcccatttttggggcaccatcgtgcagaaccaaatcgaaggcagagaactcatacttcctcatnacccttttgatctccgacttgcattggggtttggtgatgtcctgcttaataaaaatggcgcctggaattggattaacccaattcaaatccacacccacaataaggcttccacgtggcacccgtagagtggctacctgcatccaactgcctggagcagcacagaggtcgagaacagccccagttttgtgaaacaatccatac

SEQ ID NO: 4 - DNA, 403 bp, Cannabis sativa
  source 1..403: mol_type = genomic DNA
  variation 202: allele = N is T or C
tacatggaaagtatatgttgacggagcgtcaaatgaaaatggatcgggggccgaaatagcaatgatctctcctaacaggctaagactccagtctgccctacagttcacatttctggcttctaacaatgaggctgagtacaaagccctggtagcaaaattaaaattggctaaggttgtaggggccaacagggtagaaatgtanagtgattcgcaactggtggtcaaccaggtattaggggaatatcagacttggggagaaaagatggctgcttacatatcggttgttagggaattgctgcaagagtttaaggaatacaaagtggaacgaattccacgagaaagaaatgcccatgcaaactgtctggctaagctagccttagatagcgagatagagaagctgggg

SEQ ID NO: 5 - DNA, 403 bp, Cannabis sativa
  source 1..403: mol_type = genomic DNA
  variation 202: allele = N is T or G
atgggaatctcctcctaggcctttccatgaaatcccacactactaccatgaaaacatgtcaatggagtctctttgcagacttcatggatggggaattcgggaatctccaaggcgagtctatgatgcggttttattcagtaatgaagtggacatccttacaatcagatggaatgaattgtatccatatgtgacacagtttgtncttcttgagtcaaactcaactttcactggtttgccaaagcctttaattttctctggaaacagggacaaatttaagtttattgagcctcggcttacttatggtatgttggggggaaggtttcatagaggcgaaaacccttttgtggaggaggcatatcagagagtagcattggatcagcttctcagaatagcaggcatagaa

SEQ ID NO: 6 - DNA, 403 bp, Cannabis sativa
  source 1..403: mol_type = genomic DNA
  variation 202: allele = N is G or A
atctaccctttgagaaagtgaaacggaagcacgtgaagcttctgatactacagttgaaacatgatgatcagttggagatggagcctcactctgagaaaaagcgggatggctagcatctaattgaccagatcgtggccgttgaactggtccctcgagtggaaagccacccccagcagaaatagaagctagacgagtagcctcntttaaactatgaagagtgtttataagcctcaaaagtattccattctttgcagctatacgacaaaaatcatttctaggtgtagagcgctgaagcttaaagacttgccacatgccatcaatagctagatgaaccatttccctgtatgtgaagatactaatgagcaaatttcaaactgaaactataaagtattgaacaacgtac

SEQ ID NO: 7 - DNA, 403 bp, Cannabis sativa
  source 1..403: mol_type = genomic DNA
  variation 202: allele = N is T or C
tataatgatggaagcttaccagggttgattataaaagtcagaacgccgctccttctctgcatttcgactggcatctccagcaacaagctttaaatctttgctctgagaagctatcagagtgttaataaactctgctggagactgactgaaactaagaaagaaagcccgccgcctcctatgttcatgaatcttctttatagangcagatatcagttcatcgcaagcatcaatttccttattattctcagtacgtgccaagaatgaagacatttctttatccagagaaagaggcacatcaaccaacacatcatagcaattagttccaactggacaagttcccgaaagcttaatcttatgttccagatgtatgggttgtggtggagttaaatgctgtgctaacttt

SEQ ID NO: 8 - DNA, 403 bp, Cannabis sativa
  source 1..403: mol_type = genomic DNA
  variation 202: allele = N is G or A
tagttaatttgttttccagttactactaagctgcttcagcctccaataactttctttcggccaaagcatccacagcttttactttctcaggtacaaaatctacaaacatttcaagacattacatcagagagaacaataatggcataatagttgaaaaaaaatgtttaaaatcgtctatgaaaagtataaggtacctgaaagnaaatcgtatctcttccgtttgctcacaactgatgctgaaaaaacaaacttgtacttaagtactgctagcatataaatgataaaaattgttttgtaattaattgtaagaaacaattagagttaagagtaccaagttgcctaacccctctctacagtataagctgagcataaataactggtgatcctctcaagagagagaata

SEQ ID NO: 9 - DNA, 20 bp, synthetic construct
  source 1..20: mol_type = other DNA
ttgggcttccatcgaaccaa

SEQ ID NO: 10 - DNA, 20 bp, synthetic construct
  source 1..20: mol_type = other DNA
acatacccaagtggaacccc

SEQ ID NO: 11 - DNA, 20 bp, synthetic construct
  source 1..20: mol_type = other DNA
tttgggcttccatcgaacca

SEQ ID NO: 12 - DNA, 20 bp, synthetic construct
  source 1..20: mol_type = other DNA
catacccaagtggaacccca

SEQ ID NO: 13 - DNA, 20 bp, synthetic construct
  source 1..20: mol_type = other DNA
ggcatcgatgaccaaaccat

SEQ ID NO: 14 - DNA, 20 bp, synthetic construct
  source 1..20: mol_type = other DNA
aaactggggctgttctcgac

SEQ ID NO: 15 - DNA, 20 bp, synthetic construct
  source 1..20: mol_type = other DNA
aaaactggggctgttctcga

SEQ ID NO: 16 - DNA, 21 bp, synthetic construct
  source 1..21: mol_type = other DNA
ggcatcgatgaccaaaccatt

SEQ ID NO: 17 - DNA, 20 bp, synthetic construct
  source 1..20: mol_type = other DNA
gctgagtacaaagccctggt

SEQ ID NO: 18 - DNA, 20 bp, synthetic construct
  source 1..20: mol_type = other DNA
ccccagcttctctatctcgc

SEQ ID NO: 19 - DNA, 21 bp, synthetic construct
  source 1..21: mol_type = other DNA
gctgagtacaaagccctggta

SEQ ID NO: 20 - DNA, 20 bp, synthetic construct
  source 1..20: mol_type = other DNA
cccagcttctctatctcgct

SEQ ID NO: 21 - DNA, 20 bp, synthetic construct
  source 1..20: mol_type = other DNA
gcagacttcatggatgggga

SEQ ID NO: 22 - DNA, 20 bp, synthetic construct
  source 1..20: mol_type = other DNA
atgcctcctccacaaaaggg

SEQ ID NO: 23 - DNA, 20 bp, synthetic construct
  source 1..20: mol_type = other DNA
tgcagacttcatggatgggg

SEQ ID NO: 24 - DNA, 20 bp, synthetic construct
  source 1..20: mol_type = other DNA
gcctcctccacaaaagggtt

SEQ ID NO: 25 - DNA, 21 bp, synthetic construct
  source 1..21: mol_type = other DNA
agaaagtgaaacggaagcacg

SEQ ID NO: 26 - DNA, 20 bp, synthetic construct
  source 1..20: mol_type = other DNA
gcttcagcgctctacaccta

SEQ ID NO: 27 - DNA, 22 bp, synthetic construct
  source 1..22: mol_type = other DNA
gagaaagtgaaacggaagcacg

SEQ ID NO: 28 - DNA, 20 bp, synthetic construct
  source 1..20: mol_type = other DNA
gaaagtgaaacggaagcacg

SEQ ID NO: 29 - DNA, 20 bp, synthetic construct
  source 1..20: mol_type = other DNA
tggaagcttaccagggttga

SEQ ID NO: 30 - DNA, 20 bp, synthetic construct
  source 1..20: mol_type = other DNA
tgtgttggttgatgtgcctc

SEQ ID NO: 31 - DNA, 21 bp, synthetic construct
  source 1..21: mol_type = other DNA
atggaagcttaccagggttga

SEQ ID NO: 32 - DNA, 21 bp, synthetic construct
  source 1..21: mol_type = other DNA
tgtgttggttgatgtgcctct

SEQ ID NO: 33 - DNA, 20 bp, synthetic construct
  source 1..20: mol_type = other DNA
ttcggccaaagcatccacag

SEQ ID NO: 34 - DNA, 21 bp, synthetic construct
  source 1..21: mol_type = other DNA
tgtagagaggggttaggcaac

SEQ ID NO: 35 - DNA, 19 bp, synthetic construct
  source 1..19: mol_type = other DNA
ggccaaagcatccacagct

SEQ ID NO: 36 - DNA, 21 bp, synthetic construct
  source 1..21: mol_type = other DNA
actgtagagaggggttaggca

SEQ ID NO: 37 - DNA, 19 bp, synthetic construct
  source 1..19: mol_type = other DNA
ttcggccaaagcatccaca

SEQ ID NO: 38 - DNA, 30 bp, synthetic construct
  source 1..30: mol_type = other DNA
aattagcagtgttaaaatttacaacaccac

SEQ ID NO: 39 - DNA, 32 bp, synthetic construct
  source 1..32: mol_type = other DNA
aaaattagcagtgttaaaatttacaacaccat

SEQ ID NO: 40 - DNA, 30 bp, synthetic construct
  source 1..30: mol_type = other DNA
atttagctggattgatacaaccatcttcta